(19) United States
(12) Patent Application Publication
(10) Pub. No.: US 2023/0416776 A1
(43) Pub. Date: Dec. 28, 2023

(54) CRISPR-MEDIATED HUMAN GENOME EDITING WITH VECTORS

(71) Applicant: Regents of the University of Minnesota, Minneapolis, MN (US)

(72) Inventors: Li Ou, Eden Prairie, MN (US); Chester B. Whitley, Brooklyn Park, NY (US)

(21) Appl. No.: 17/767,540

(22) PCT Filed: Oct. 8, 2020
(86) PCT No.: PCT/US2020/054835

§ 371 Date: Apr. 8, 2022

Related U.S. Application Data
(60) Provisional application No. 62/912,329, filed on Oct. 8, 2019.

(51) Int. Cl. C12N 15/86; C12N 15/11; C12N 9/22

(52) U.S. Cl. CPC C12N 15/86; C12N 15/11; C12N 2830/008; C12N 2310/20; C12N 2750/14143; C12N 9/22

(57) ABSTRACT

Compositions and methods for Cas-based ex vivo and in vivo gene therapy applications are provided.

### CROSS-REFERENCE TO RELATED APPLICATIONS

This application claims the benefit of the filing date of U.S. application No. 62/912 29, filed on Oct. 8, 2019, the disclosure of which is incorporated by reference herein.

### BACKGROUND

Gene therapy holds enormous potential for a new era of human therapeutics. These methodologies will allow treatment for conditions that heretofore have not been addressable by standard medical practice. One area that is especially promising is the ability to add a transgene to a cell to cause that cell to express a product that previously not being produced (or produced at insufficient levels) in that cell. Examples of uses of this technology include the insertion of a gene encoding a therapeutic protein, insertion of a coding sequence encoding a protein that is somehow lacking in the cell or in the individual and insertion of a sequence that encodes a structural nucleic acid such as a microRNA or siRNA.

Transgenes can be delivered to a cell by a variety of ways, such that the transgene becomes integrated into the cell's own genome and is maintained there. In recent years, a strategy for transgene integration has been developed that uses cleavage with site-specific nucleases for targeted insertion into a chosen genomic locus (see, e.g., U.S. Pat. No. 7,888,121). Nucleases, such as zinc finger nucleases (ZFNs), transcription activator-like effector nucleases (TALENs), or nuclease systems such as the CRISPR/Cas system (utilizing an engineered guide RNA), are specific for targeted genes and can be utilized such that the transgene construct is inserted by either homology directed repair (HDR) or by end capture during non-homologous end joining (NHEJ) driven processes.

### SUMMARY

In one embodiment, the invention provides for delivery of one or more genes encoding proteins using CRISPR/Cas, delivered via one or more vectors such as plasmids or viral vectors, including but not limited to lentivirus vectors, adenovirus vectors, adeno-associated virus (AAV) vectors, e.g., AAV2, AAV5, AAV6, AAV8, or AAV9, or herpesvirus vectors, which proteins may be useful to prevent, inhibit or treat diseases such as monogenic diseases, e.g., lysosomal storage diseases, hemophilia, thalassemia, sickle cell diseases and the like. In one embodiment, at least one or two vectors are used to deliver one or more CRISPR components, e.g., nucleic acid encoding Cas, gRNA(s), a gene encoding the protein or interest, e.g., which is optionally promoterless, for targeted insertion into the genome of a human cell, e.g., ex vivo or in vivo. In one embodiment, systemic of the one or more vectors administration is employed. In one embodiment, Cas may be supplied in trans. Combinations of different vectors and/or proteins may be used. Sequences for gRNA and homology arms flanking the gene of interest may be directed to any insertion (target) site in the genome of a human cell so long as the site allows for adequate expression of the introduced gene. Exemplary insertion sites include but are not limited to the albumin locus, AAVS1, Rosa26, CCR5, HPRT, or the alpha fetoprotein locus, e.g., intron 1 of the albumin locus, AAVS1, Rosa26, CCR5, HPRT, or the alpha fetoprotein locus. In one embodiment, a human genome site (a locus) for insertion of a gene of interest has few if any polymorphisms, e.g., selected gRNA(s) and/or homology arm sequences are useful for more than one individual as the sequences at and near the insertion site are conserved among genetically unrelated individuals. In one embodiment, the gRNA sequence is directed to a conserved sequence. In one embodiment, where the locus is polymorphic, the gRNA sequence may be directed to a conserved sequence and the homology arms may have a polymorphic sequence, e.g., the homology arms may be specific for an individual. In one embodiment, where the locus is polymorphic, the gRNA sequence and the homology arms may have polymorphic sequences, e.g., both the gRNA and the homology arms are specific for an individual. In one embodiment, the vector(s) is/are mRNA, e.g., in a nanoparticle such as a liposome. In one embodiment, the vector(s) is/are plasmid vectors, e.g., in a nanoparticle such as a liposome. In one embodiment, the vector(s) is/are viral vectors. In one embodiment, the viral vectors is an adeno-associated virus vector. In one embodiment, one vector is employed. In one embodiment, two vectors are employed.

In one embodiment, a method to prevent, inhibit or treat a disease in a mammal or a mammalian cell is provided. The method includes administering an effective amount of i) Cas or an isolated nucleic encoding Cas, e.g., a vector comprising an isolated nucleic encoding Cas, and ii) isolated nucleic acid for one or more gRNAs comprising a targeting sequence for a genomic target and nucleic acid comprising a coding sequence for a prophylactic or therapeutic gene product flanked by homology arms, e.g., a vector comprising isolated nucleic acid for one or more gRNAs comprising a targeting sequence for a genomic target and nucleic acid comprising a coding sequence for a prophylactic or therapeutic gene product flanked by homology arms, or an effective amount of iii) isolated nucleic encoding Cas and nucleic acid for one or more gRNAs comprising a targeting sequence for a genomic target, e.g., a vector comprising isolated nucleic encoding Cas and nucleic acid for one or more gRNAs comprising a targeting sequence for a genomic target, and iv) isolated nucleic acid comprising a coding sequence for a prophylactic or therapeutic gene product flanked by homology arms, e.g., a vector comprising isolated nucleic acid comprising a coding sequence for a prophylactic or therapeutic gene product flanked by homology arms, wherein the expression of the coding sequence in the mammal prevents, inhibits or treats the disease or in the mammalian cell results in increased expression of the prophylactic or therapeutic gene product. In one embodiment, the mammal is a human. In one embodiment, at least one homology arm has one or more mutations that decrease subsequent cleavage events by the introduced recombinase, e.g., Cas9. in one embodiment, a composition comprises Cas9 or an isolated nucleic encoding Cas9, and isolated nucleic acid for one or more gRNAs comprising a targeting sequence for a genomic target and nucleic acid comprising a coding sequence for a prophylactic or therapeutic gene product flanked by homology arm. In one embodiment, the Cas is SpCas9. In one embodiment, the Cas is SaCas. In one embodiment, a composition comprises isolated nucleic encoding Cas9 and nucleic acid for one or more gRNAs comprising a targeting sequence for a genomic target, and isolated nucleic acid comprising a coding sequence for a prophylactic or therapeutic gene product flanked by homology arms. In one embodiment, the targeting sequence targets intron 1 of the albumin locus. In one embodiment, the targeting sequence comprises at least 20 contiguous nucleotides in intron 1 of the albumin locus. In one embodiment, the targeting sequence comprises at least 20, 25, 30, 35 or 40 contiguous nucleotides in one of

(SEQ ID NO: 20)
tccatttttc tattgttcaa cttttattct
attttcccag taaaataaag ttttagtaaa
ctctgcatct ttaaagaatt attttggcat
ttatttctaa aatggcatag tattttgtat
ttgtgaagtc ttacaaggtt atcttattaa
taaaattcaa acatcctagg taaaaaaaaa
aaaaggtcag aattgtttag tgactgtaat
tttcttttgc gcactaagga aagtgcaaag
taacttagag tgactgaaac ttcacagaat
agggttgaag attgaattca taactatccc
aaagacctat ccattgcact atgctttatt
taaaaaccac aaaacctgtg ctgttgatct
cataaataga acttgtattt atatttattt
tcattttagt ctgtcttctt ggttgctgtt
gatagacact aaaagagtat tagatattat
ctaagtttga atataaggct ataaatattt
aataattttt aaaatagtat tcttggtaat
tgaattattc ttctgtttaa aggcagaaga
aataattgaa catcatcctg agtttttctg
taggaatcag agcccaatat tttgaaacaa
(SEQ ID NO: 21)
ttgtgaagtc ttacaaggtt atcttattaa
taaaattcaa acatcctagg tasasaaaaa
aaaaggtcag aattgtttag tgactgtaat
tttcttttgc gcactaagga aagtgcaaag
taacttagag tgactgaaac ttcacagaat
agggttgaag attgaattca taactatccc
aaagacctat ccattgcact atgctttatt
taaaaaccac aaaacctgtg ctgttgatct
cataaataga acttgtattt atatttattt
tcattttagt ctgtcttctt ggttgctgtt
gatagacact aaaagagtat tagatattat
ctaagtttga atataaggct ataaatattt
(SEQ ID NO: 22)
taacttagag tgactgaaac ttcacagaat
agggttgaag attgaattca taactatccc
aaagacctat ccattgcact atgctttatt
taaaaaccac aaaacctgtg ctgttgatct
cataaataga acttgtattt atatttattt
tcattttagt ctgtcttctt ggttgctgtt
gatagacact aaaagagtat tagatattat
ctaagtttga atataaggct ataaatattt
aataattttt aaaatagtat tcttggtaat
tgaattattc ttctgtttaa aggcagaaga

or the complement thereof. In one embodiment, the targeting sequence begins 400, 425, 410, 420, 425, 428, 430 or more nucleotides downstream of the ATG (start) codon for albumin. In one embodiment, a Cas9 or an isolated nucleic encoding Cas9 and isolated nucleic acid for one or more gRNAs comprising a targeting sequence for a genomic target and nucleic acid comprising a coding sequence for a prophylactic or therapeutic gene product flanked by homology arm are separately administered, e.g., sequentially or at different locations. In one embodiment, isolated nucleic encoding Cas9 and nucleic acid for one or more gRNAs comprising a targeting sequence for a genomic target and isolated nucleic acid comprising a coding sequence for a prophylactic or therapeutic gene product flanked by homology arms are separately administered, e.g., sequentially or at different locations. In one embodiment, a Cas9 or an isolated nucleic encoding Cas9 and isolated nucleic acid for one or more gRNAs comprising a targeting sequence for a genomic target and nucleic acid comprising a coding sequence for a prophylactic or therapeutic gene product flanked by homology arm are administered at the same time and at the same location. In one embodiment, isolated nucleic encoding Cas9 and nucleic acid for one or more gRNAs comprising a targeting sequence for a genomic target and isolated nucleic acid comprising a coding sequence for a prophylactic or therapeutic gene product flanked by homology arms are administered at the same time and at the same location. In one embodiment, the disease is mucopolysaccharidosis, a lysosomal storage disease, hemophilia, thalassemia, or sickle cell disease. In one embodiment, the targeting sequence or homology arms are targeted to an intron. In one embodiment, one or more adeno-associated virus (AAV), adenovirus or lentivirus is/are employed to deliver at least one of Cas9 or an isolated nucleic encoding Cas9, or isolated nucleic acid for one or more gRNAs comprising a targeting sequence for a genomic target and nucleic acid comprising a coding sequence for a prophylactic or therapeutic gene product flanked by homology arms, or at least one of isolated nucleic encoding Cas9 and nucleic acid for one or more gRNAs comprising a targeting sequence for a genomic target, or isolated nucleic acid comprising a coding sequence for a prophylactic or therapeutic gene product flanked by homology arms. In one embodiment, a first rAAV delivers nucleic acid encoding Cas9. In one embodiment, a second rAAV delivers the nucleic acid comprising the targeting sequence and the coding sequence. In one embodiment, the first or second AAV is one of serotypes AAV1-9 or AAVrh10. In one embodiment, the first and the second rAAVs are different serotypes. In one embodiment, the mammal is a human. In one embodiment, one or more of the gRNAs target the albumin locus, the Rosa26 locus, AAVS1 locus, CCR5 locus, HPRT locus, or alpha fetoprotein locus. In one embodiment, the disease is mucopolysaccharoidosis type I, type II type III, type IV, type V, type VI or type VII. In one embodiment, the disease is Tay-Sachs disease or Sandhoff disease (GM2-gangliosidosis disease). In one embodiment, the coding sequence encodes iduronidase, beta-globin, iduronate, beta galactosidase, sulfatase, hexM, hexoaminidase A or hexosaminidase B. In one embodiment, the intron is an albumin gene intron. In one embodiment, the intron is the first intron. In one embodiment, the targeting sequence is promoterless, e.g., until inserted into the host cell genome. In one embodiment, the targeting sequence targets sequences within the first 500, 400, 300, 200, or 100 nucleotides of the intron. In one embodiment, the Cas9 comprises Streptococcus pyogenes (SpCas9), Staphylococcus aureus (SaCas9), Streptococcus thermophiles (StCas9), Neisseria meningitidis (NmCas9), Francisella novicida (FnCas9), Campylobacter jejuni (CjCas9), CasX, CasY, Cas12a (Cpf1), Cas14a, eSpCas9, SpCas9-HF1, HypaCas9, Fokl-Fused dCas9, or xCas9. In one embodiment, liposomes are employed to deliver Cas9 or an isolated nucleic encoding Cas9, isolated nucleic acid for one or more gRNAs comprising a targeting sequence for a genomic target and nucleic acid comprising a coding sequence for a prophylactic or therapeutic gene product flanked by homology arms, isolated nucleic encoding Cas9 and nucleic acid for one or more gRNAs comprising a targeting sequence for a genomic target, isolated nucleic acid comprising a coding sequence for a prophylactic or therapeutic gene product flanked by homology arms, or any combination thereof. In one embodiment, the nucleic acid comprising a coding sequence for a prophylactic or therapeutic gene product is not operably linked to a promoter. In one embodiment, at least one of Cas9 or an isolated nucleic encoding Cas9, isolated nucleic acid for one or more gRNAs comprising a targeting sequence for a genomic target and nucleic acid comprising a coding sequence for a prophylactic or therapeutic gene product flanked by homology arms, isolated nucleic encoding Cas9 and nucleic acid for one or more gRNAs comprising a targeting sequence for a genomic target, or isolated nucleic acid comprising a coding sequence for a prophylactic or therapeutic gene product flanked by homology arms is delivered parenterally. In one embodiment, at least one of Cas9 or an isolated nucleic encoding Cas9, isolated nucleic acid for one or more gRNAs comprising a targeting sequence for a genomic target and nucleic acid comprising a coding sequence for a prophylactic or therapeutic gene product flanked by homology arms, isolated nucleic encoding Cas9 and nucleic acid for one or more gRNAs comprising a targeting sequence for a genomic target, or isolated nucleic acid comprising a coding sequence for a prophylactic or therapeutic gene product flanked by homology arm is delivered intravenously. For example, Cas protein may be delivered via a different route that one of the isolated nucleic acids. In one embodiment, a single administration is effective to prevent, inhibit or treat a disease, or one or more symptoms thereof, in a mammal. In one embodiment, a dose of virus may be from about 1×10¹² vg/kg to about 1×10¹⁴ vg/kg, e.g., about 3×10¹² vg/kg to about 5×10¹³ vg/kg. In one embodiment, the ratio of Cas vector to the donor vector is about 1:20, 1:15, 1:10, 1:8, 1:6, 1:5, 1:2 or 1:1. In one embodiment, the ratio of Cas encoding viral particles to donor nucleic acid containing viral particles is about 1:20, 1:15, 1:10, 1:8, 1:6, 1:5, 1:2 or 1:1.

Further provided is a composition comprising a first rAAV comprising an isolated nucleic encoding Cas, e.g., SpCas9, and a second rAAV comprising an isolated nucleic comprising sequences for one or more gRNAs comprising a selected targeting sequence, e.g., targeted to intron 1 of the human albumin locus, and a selected coding sequence flanked by homology arms, e.g., at least one of which arms is mutated relative to the genomic sequence in the human, or a first rAAV comprising an isolated nucleic encoding Cas, e.g., SpCas9, and an isolated nucleic comprising sequences for one or more gRNAs comprising a selected targeting sequence, e.g., targeted to intron 1 of the human albumin locus, and a second rAAV comprising a selected coding sequence flanked by homology arms, e.g., at least one of which arms is mutated relative to the genomic sequence in the human, e.g., a homology arm may have from about 15 to 200, 50 to 80, 50 to 100, 100 to 150, 100 to 200, 200 to 500, 300 to 500, 500 to 1000, 1000 to 2000, or 2500 or more, nucleotides in length. In one embodiment, the homology arm that is mutated has 1, 2, 3, 4, 5, 6, 7, 8, 9, 10, 11, 12, 13 or more mutations, e.g., every other nucleotide is mutated, or every other nucleotide is mutated for about 10 to 15 nucleotides, then two consecutive nucleotides are mutated, or intermittently, every other nucleotide is mutated, two consecutive nucleotides are mutated, two consecutive nucleotides are not mutated, or any combination thereof, relative to the target site. In one embodiment, the homology arm that is mutated has 10, 20, 30, 40, 50, 60, or 70% of its nucleotides mutated relative to the target site. If the insertion site is in a coding region, in one embodiment, the mutations do not alter the encoded amino acid(s).

In one embodiment, one or more CRISPR components and the gene of interest are delivered using viral vectors, e.g., one or more lentivirus vectors or two rAAV vectors. In one embodiment, the rAAV vector is a rAAV2, rAAV5, rAAV6, rAAV8, or rAAV9 vector. In one embodiment, the rAAVs are administered to an embryo, a fetus, an infant (e.g., a human that is 3 years old or less such as less than 3, 2.5, 2, or 1.5 years of age), a pre-adolescent (e.g., in humans those less than 10, 9, 8, 7, 6, 5, or 4 but greater than 3 years of age), or adult (e.g., humans older than about 12 years of age).

In one embodiment, the mammal is a human. In one embodiment, multiple doses are administered. In one embodiment, the composition is administered weekly, monthly or two or more months apart. In one embodiment, a single dose is administered.

In one embodiment, the amount of vector(s) administered results in an increase, e.g., at least 2-, 5-, 10-, 25-, 50-, 100-, 200- or 500-fold or more, up to 1000-fold of the gene product, e.g., in plasma or tissue, e.g., the brain, in the mammal relative to a corresponding mammal with that is not administered the vectors.

Diseases that may be prevented, inhibited or treated using the methods disclosed herein include, but are not limited to, Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy (a deficiency of survivor of motor neuron-1, SMN-1), Spinocerebellar ataxia, Tangier disease, Tay-Sachs disease, Tuberous sclerosis, Von Hippel-Lindau syndrome, Williams syndrome, Wilson's disease, or Zellweger syndrome. In one embodiment, the disease is a lysosomal storage disease, e.g., a lack or deficiency in a lysosomal storage enzyme. Lysosomal storage diseases include, but are not limited to, mucopolysaccharidosis (MPS) diseases, for instance, mucopolysaccharidosis type I, e.g., Hurler syndrome and the variants Scheie syndrome and Hurler-Scheie syndrome (a deficiency in alpha-L-iduronidase); Hunter syndrome (a deficiency of iduronate-2-sulfatase); mucopolysaccharidosis type III, e.g., Sanfilippo syndrome (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV, e.g., Morquio syndrome (a deficiency of galactosamine-6-sulfate sulfatase or beta-galactosidase); mucopolysaccharidosis type VI, e.g., Maroteaux-Lamy syndrome (a deficiency of arylsulfatase B); mucopolysaccharidosis type II; mucopolysaccharidosis type III (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV (A or B; a deficiency of galactosamine-6-sulfatase and beta-galatacosidase); mucopolysaccharidosis type VI (a deficiency of arylsulfatase B); mucopolysaccharidosis type VII (a deficiency in beta-glucuronidase); mucopolysaccharidosis type VIII (a deficiency of glucosamine-6-sulfate sulfatase); mucopolysaccharidosis type IX (a deficiency of hyaluronidase); Tay-Sachs disease (a deficiency in alpha subunit of beta-hexosaminidase); Sandhoff disease (a deficiency in both alpha and beta subunit of beta-hexosaminidase); GM1 gangliosidosis (type I or type II); Fabry disease (a deficiency in alpha galactosidase); metachromatic leukodystrophy (a deficiency of aryl sulfatase A); Pompe disease (a deficiency of acid maltase); fucosidosis (a deficiency of fucosidase); alpha-mannosidosis (a deficiency of alpha-mannosidase); beta-mannosidosis (a deficiency of beta-mannosidase), neuronal ceroid lipofuscinosis (NCL) (a deficiency of ceroid lipofucinoses (CLNs), e.g., Batten disease having a deficiency in the gene product of one or more of CLN1 to CLN14), and Gaucher disease (types I, II and III; a deficiency in glucocerebrosidase), as well as disorders such as Hermansky-Pudlak syndrome; Amaurotic idiocy; Tangier disease; aspartylglucosaminuria; congenital disorder of glycosylation, type Ia; Chediak-Higashi syndrome; macular dystrophy, corneal, 1; cystinosis, nephropathic; Fanconi-Bickel syndrome; Farber lipogranulomatosis; fibromatosis; geleophysic dysplasia; glycogen storage disease I; glycogen storage disease Ib; glycogen storage disease Ic; glycogen storage disease III; glycogen storage disease IV; glycogen storage disease V; glycogen storage disease VI; glycogen storage disease VII; glycogen storage disease 0; immunoosseous dysplasia, Schimke type; lipidosis; lipase b; mucolipidosis II; mucolipidosis II, including the variant form; mucolipidosis IV; neuraminidase deficiency with beta-galactosidase deficiency; mucolipidosis I; Niemann-Pick disease (a deficiency of sphingomyelinase); Niemann-Pick disease without sphingomyelinase deficiency (a deficiency of a npc1 gene encoding a cholesterol metabolizing enzyme); Refsum disease; Sea-blue histiocyte disease; infantile sialic acid storage disorder; sialuria; multiple sulfatase deficiency; triglyceride storage disease with impaired long-chain fatty acid oxidation; Winchester disease; Wolman disease (a deficiency of cholesterol ester hydrolase); Deoxyribonuclease I-like 1 disorder; arylsulfatase E disorder; ATPase, H+ transporting, lysosomal, subunit 1 disorder; glycogen storage disease IIb; Ras-associated protein rab9 disorder; chondrodysplasia punctata 1, X-linked recessive disorder; glycogen storage disease VIII; lysosome-associated membrane protein 2 disorder; Menkes syndrome; congenital disorder of glycosylation, type Ic; and sialuria. Replacement of less than 20%, e.g., less than 10% or about 1% to 5% levels of lysosomal storage enzyme found in nondiseased mammals, may prevent, inhibit or treat neurological symptoms such as neurological degeneration in mammals. In one embodiment, the disease to be prevented, inhibited or treated with a particular gene includes, but is not limited to, MPS I (IDUA), MPS II (IDS), MPS IIIA (Heparan-N-sulfatase; sulfaminidase), MPS IIIB (alpha-N-acetyl-glucosaminidase), MPS IIIC (Acetyl-CoA:alpha-N-acetyl-glucosaminide acetyltransferase), MPS IIID (N-acetylglucosamine 6-sulfatase), MPS VII (beta-glucoronidase), Gaucher (acid beta-glucosidase), Alpha-mannosidosis (alpha-mannosidase), Beta-mannosidosis (beta-mannosidase) Alpha-fucosidosis (alpha-fucosidase), Sialidosis (alpha-sialidase), Galactosialidosis (Cathepsin A), Aspartylglucosaminuria (aspartylglucosaminidase), GM1-gangliosidosis (beta-galactosidase), Tay-Sachs (beta-hexosaminidase subunit alpha), Sandhoff (beta-hexosaminidase subunit beta), GM2-gangliosidosis/variant AB (GM2 activator protein), Krabbe (galactocerebrosidase), Metachromatic leukodystrophy (arylsulfatase A), hemophilia (factor VIII or factor IX), thalassemia (HBB, HBA1, or HBA2), sickle cell anemia (HBB), von Willenbrand disease (von Willenbrand factor), and other disorders including but not limited to Alzheimer's disease (expression of an antibody, such as an antibody to beta-amyloid, or an enzyme that attacks the plaques and fibrils associated with Alzheimer's), or Alzheimer's and Parkinson's diseases (expression of neuroprotective proteins including but not limited to GDNF or Neurturin). In one embodiment, the gene encodes factor VIII. In one embodiment, the gene encodes factor IX, In one embodiment, the gene encodes beta-globin. In one embodiment, the gene encodes alpha-globin.

In one embodiment, the disclosure provides for use of i) Cas or an isolated nucleic encoding Cas and isolated nucleic acid for one or more gRNAs comprising a targeting sequence for a human albumin genomic target and nucleic acid comprising a coding sequence for a prophylactic or therapeutic gene product flanked by homology arms that bind to the human genomic target, or ii) isolated nucleic encoding Cas and nucleic acid for one or more gRNAs comprising a targeting sequence for a human albumin genomic target and isolated nucleic acid comprising a coding sequence for a prophylactic or therapeutic gene product flanked by homology arms that bind to the human genomic target. In one embodiment, the use is gene therapy.

### BRIEF DESCRIPTION OF THE FIGURES

FIG. 1. Construct design. Sequence of AAV vectors represented in cartoon. hAAT: human α1-antitrypsin promoter; ITR: inverted terminal repeats; SA: splice acceptor; SD; splice donor; PA: poly A; HA: homology arm; IDUA: human IDUA cDNA; RE: restriction enzyme site; U6: U6 promoter sequence.

FIG. 2. Exemplary vectors. hAAT: human a1-antitrypsin promoter; TBG: thyroxine-binding globulin; ITR: inverted terminal repeats; SA: splice acceptor; SD; splice donor; PA: polyA; HA: homology arm; RE: restriction enzyme site; U6: U6 promoter sequence.

FIG. 3. Exemplary vectors and promoters.

FIG. 4. Gene editing can avoid the vector dilution issue associated with AAV gene therapy. The episomal AAV vector in transduced cells is diluted during each round of cell division. In contrast, the edited sequence can be replicated and remain after cell divisions, thus avoiding vector dilution.

FIG. 5. Molecular mechanism of the PS gene editing system. First, Cas9 nuclease creates a double strand break at the target locus. Through homology directed repair, the splicing acceptor (SA) and human IDUA cDNA (IDUA), and polyA (PA) sequence are integrated. Through non-homologous end joining, the whole AAV sequence is integrated. Through the interaction between splicing donor (SD) and SA, the fusion transcript is the same, which includes the exon 1 of albumin gene, and IDUA sequence. The exon 1 primarily encodes a signal peptide, which will be cleaved thereafter. The mature proteins are only the therapeutic IDUA proteins. ITR: inverted terminal repeat; HA: homology arm.

FIG. 6A-C. The cutting efficiency and specificity of PS822. (A) Plasmids encoding SpCas9 and 3 candidate sgRNAs were transfected into human hepatocytes (Huh-7 cell line), and the cleavage activity was measured through sequencing the target locus. Only sgRNA3 showed significant cleavage. (B) SpCas9 and sgRNA3 ribonucleoprotein were cotransfected with double strand oligo tag (dsTag) into Huh-7 cells. Genomic DNA was extracted and used for library preparation. Deep sequencing was performed to search for the dsTag. (C) Only on-target cleavage was identified through GUIDE-seq.

FIG. 7A-B. Enzyme activities and GAG levels in MPS I mice after treatment with the mouse PS822 surrogate reagents. (A) Enzyme activities increased significantly, and GAG storage levels (B) reduced significantly in tissues including the brain at 11 month post dosing. Mean±SEM. *p<0.05 when comparing treated to untreated MPS I mice, **p<0.01, ***p<0.001, ****p<0.0001, One-way ANOVA for multiple comparisons.

FIG. 8A-D. Additional pharmacology outcomes in MPS I mice treated with the mouse PS822 surrogate reagents. (A) Blood samples were collected from all mice monthly. Plasma IDUA enzyme activity increased significantly throughout 10 months. (B) Kaplan-Meier analysis showed the improved survival rate of treated mice. (D) Tumor risk was not significantly increased in treated mice. (C) Fear conditioning showed treated mice had better memory and learning ability. (D) Pole test showed that treated MPS I mice had better motor function. Mean±SEM. *p<0.05 when comparing treated to untreated MPS I mice, **p<0.01, ***p<0.001, ****p<0.0001. One-way ANOVA for multiple comparisons, Log-rank (Mantel-Cox) test for survival analysis.

FIG. 9A-C. Pharmacology outcomes in SD mice treated with the PS system. (A) Hex A enzyme activities in plasma significantly increased at 1, 2 and 3 months postdosing. (B) Hex A enzyme activities In tissues including in the brain increased significantly, (C) Treated SD mice had better performance in the rotarod test at 4 month post dosing, *p<:0.05 when compared with untreated SD mice.

FIG. 10. Histological analysis of SD mice treated with the PS system. (A) The brain and liver were processed for H&E staining (upper and middle panel), and immunohistochemisty for Hex A enzyme (lower panel). Treated SD mice, untreated SD and normal mice are shown in the left, middle and right columns, respectively. Kupffer cell vacuolation (small, well defined, vesicles with clear to pale-eosinophilic content) in the liver of untreated SD mice was reduced in treated SD mice. In the cerebellum, pons, thalamus, hypothalamus and brain cortex of untreated SD mice, there was neuronal vacuolation, which was minimal to mild in treated SD and normal mice. When the brain was stained against Hex A proteins, the signal intensity in treated SD mice was comparable to normal mice, while only minimal signal was observed in untreated SD mice. Objective x40.

FIGS. 11A-11B. cDNA integration at the human albumin locus and transgene expression. (A) The sgRNA3 that mediates efficient cleavage at the human albumin locus was packaged into the donor plasmid that encodes IDUA cDNA. Then, the donor plasmid was cotransfected with the plasmid encoding SpCas9 into HepG2 cells. After 48 hours, genome DNA was extracted from collected cells. Nested PCR was performed with two sets of primers: Nested1-F (5′-TATACACAAGGGATTTAGTCAAAC-3′) (SEQ ID NO:23) and Nested1-R (5′-TGGGTAAGCCACCAAAGGAAAC-3′) (SEQ ID NO:24); Nested2-F (5′-GGCAGCCAATGAAATACAAAGAT-3′) (SEQ ID NO:25) and Nested2-R 5′-ACCAGGTCCTTCCACTCGAACA-3′) (SREQ ID NO:26). The amplicons were confirmed by sequencing. (B) Cell pellets and supernatants were also collected from IDUA enzyme assays. Hepatocytes transfected with Cas9 and cDNA donor had significantly higher enzyme activity than controls, indicating successful transgene expression of therapeutic proteins.

FIG. 12. Target albumin sites and PAM sequences for SaCas9.

FIGS. 13A-13B. A) Target albumin sites and PAM sequences for SpCas9. Sequences in blue: complementary to the Bbsl-digested vector Yellow highlighted sequence: G added. B) Left homology arm and region for mutations therein.

FIG. 14. Polymorphisms at an exemplary target locus.

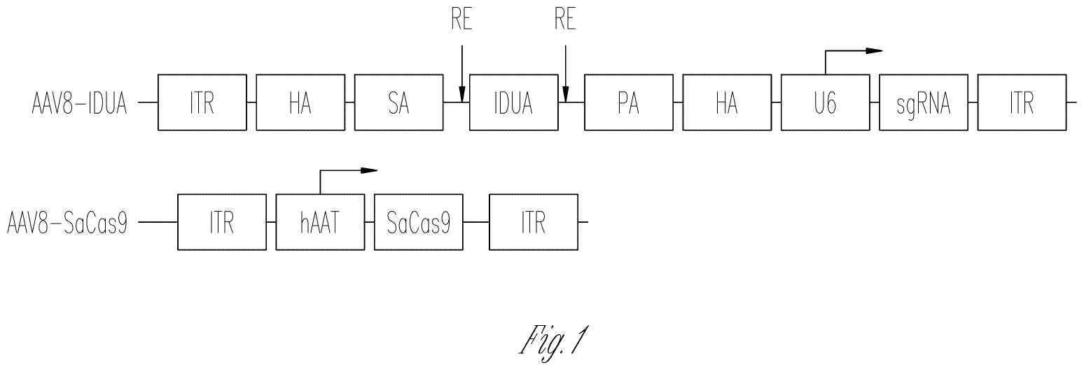

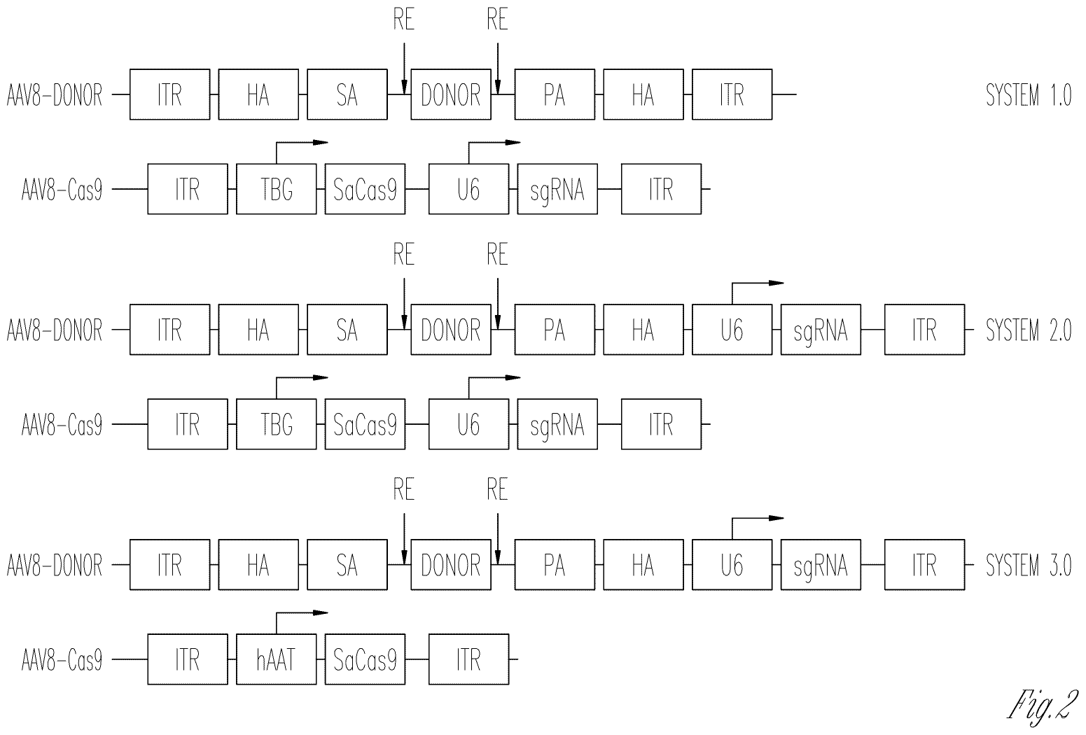

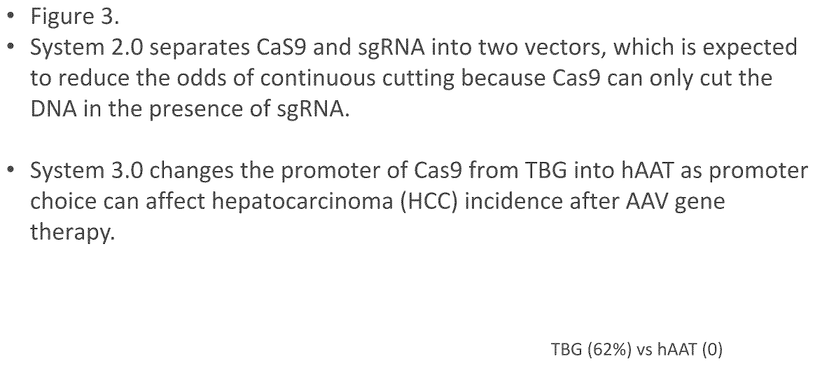

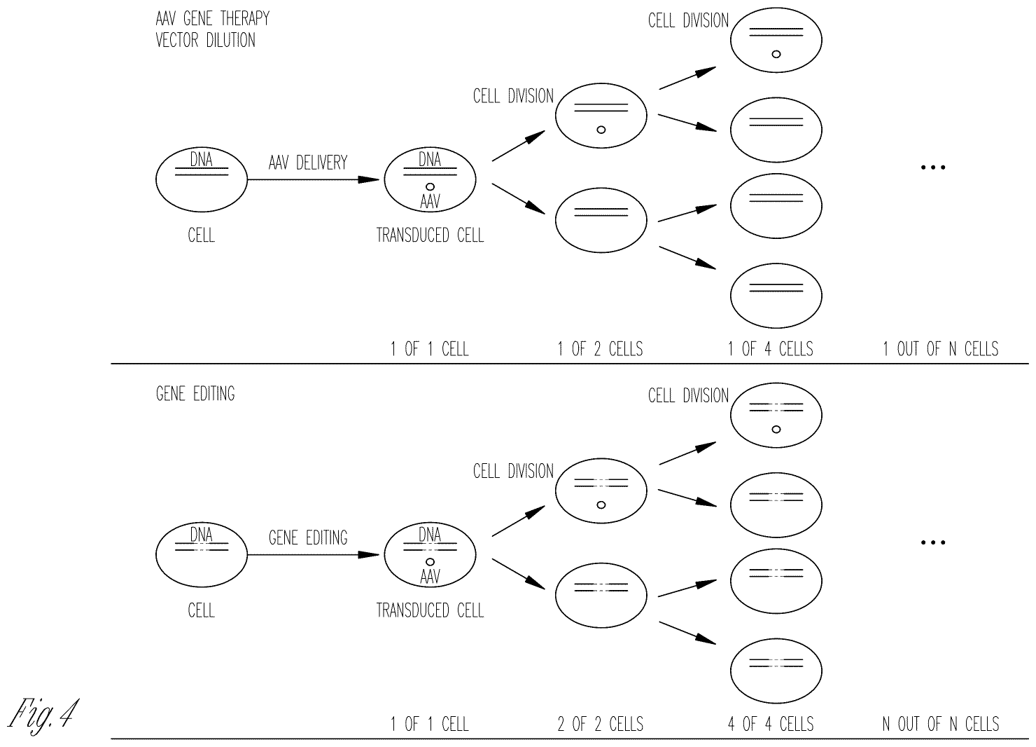

### DETAILED DESCRIPTION

### Definitions

As used herein. “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably, and are used to refer to diseases or conditions wherein lack of or reduced amounts of a specific gene product, e.g., a lysosomal storage enzyme, plays a role in the disease such that a therapeutically beneficial effect can be achieved by supplementing, e.g., to at least 1% of normal levels.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, e.g., a recombinant AAV encoding a gene product, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s)are outweighed by the therapeutically beneficial effects.

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest) and/or a selectable or detectable marker.

“AAV” is adeno-associated virus, and may be used to refer to the virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its binding properties, e.g., there are eleven serotypes of AAVs, AAV1-AAV11, including AAV2, AAV5, AAV6, AAV8, AAV9 and AAVrh10, and the term encompasses pseudotypes with the same binding properties. Thus, for example, AAV9 serotypes include AAV with the binding properties of AAV9, e.g., a pseudotyped AAV comprising AAV9 capsid and a rAAV genome which is not derived or obtained from AAV9 or which genome is chimeric. The abbreviation “rAAV” refers to recombinant adeno-associated virus, also referred to as a recombinant AAV vector (or “rAAV vector”).

An “AAV virus” refers to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”. An AAV “capsid protein” includes a capsid protein of a wild-type AAV, as well as modified forms of an AAV capsid protein which are structurally and or functionally capable of packaging a rAAV genome and bind to at least one specific cellular receptor which may be different than a receptor employed by wild type AAV, A modified AAV capsid protein includes a chimeric AAV capsid protein such as one having amino acid sequences from two or more serotypes of AAV, e.g., a capsid protein formed from a portion of the capsid protein from AAV9 fused or linked to a portion of the capsid protein from AAV-2, and a AAV capsid protein having a tag or other detectable non-AAV capsid peptide or protein fused or linked to the AAV capsid protein, e.g., a portion of an antibody molecule which binds a receptor other than the receptor for AAV9, such as the transferrin receptor, may be recombinantly fused to the AAV9 capsid protein.

A “pseudotyped” rAAV is an infectious virus having any combination of an AAV capsid protein and an AAV genome. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 inverted terminal repeats (ITRs), each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric. i.e., an ITR which has ITR sequences from more than one AAV serotype.

The terms “nucleic add,” “polynucleotide,” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. “Affinity” refers to the strength of binding: increased binding affinity being correlated with a lower Kd.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

A “disease associated gene” is one that is defective in some manner in a monogenic disease. Non-limiting examples of monogenic diseases include severe combined immunodeficiency, cystic fibrosis, lysosomal storage diseases (e.g. Gaucher's, Hurler's Hunter's, Fabry's, Neimann-Pick, Tay-Sach's etc), sickle cell anemia, and thalassemia.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence.

### The CRISPR/Cas System

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system. The primary products of the CRISPR loci appear to be short RNAs that contain the invader targeting sequences, and are termed guide RNAs

“Cas1” polypeptide refers to CRISPR associated (Cas) protein). Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cast polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cas1 polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cas1 polypeptide is a Cas1 polypeptide that is a member of CASS3 or CASS7.

In some embodiments, a Cas1 polypeptide is encoded by a nucleotide sequence provided in GenBank at, e.g., GeneID number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins, Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short moth in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013)

Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof.

### RNA Components of CRISPR/Cas

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA, (conferring target specificity) is fused to a tracrRNA (supplying interaction with the Cas9) to create a chimeric cr-RNA-tracrRNA hybrid (also termed a single guide RNA), (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[n20]GG formula.

### Donors

As noted above, insertion of an exogenous sequence (also called a “donor sequence” or “donor” or “transgene” or “gene of interest”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang, et al. (1987) Proc. Natl. Acad. Sci. USA 84:4959-4963; Nehis, et al. (1996) Science 272:886-889, Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the lysosomal sequences. In other embodiments, the transgene (e.g., with or without additional coding sequences such as for albumin) is integrated into any endogenous locus, for example a safe-harbor locus. See, e.g., U.S. Patent Publication Nos. 2008/0299580; 2008/0159996; and 2010/0218264.

When endogenous sequences (endogenous or part of the transgene) are expressed with the transgene, the endogenous sequences (e.g., albumin, etc.) may be full-length sequences (wild-type or mutant) or partial sequences. Preferably the endogenous sequences are functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

### Exemplary rAAV Vectors

Adeno-associated viruses of any serotype are suitable to prepare rAAV, since the various serotypes are functionally and structurally related, even at the genetic level. All AAV serotypes apparently exhibit similar replication properties mediated by homologous rep genes; and all generally bear three related capsid proteins such as those expressed in AAV2. The degree of relatedness is further suggested by heteroduplex analysis which reveals extensive cross-hybridization between serotypes along the length of the genorne; and the presence of analogous self-annealing segments at the termini that correspond to ITRs. The similar infectivity patterns also suggest that the replication functions in each serotype are under similar regulatory control. Among the various AAV serotypes, AAV2 is most commonly employed.

An AAV vector of the invention typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and may be preferred when it is desired that the therapeutic or prophylactic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, e.g., (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present invention.

The native promoters for rep are sell-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters may be preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1CP4; as well as vaccinia or poxvirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), e.g., linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Removal of one or more AAV genes is in any case desirable, to reduce the likelihood of generating replication-competent AAV (“RCA”). Accordingly, encoding or promoter sequences for rep, cap, or both, may be removed, since the functions provided by these genes can be provided in trans, e.g., in a stable line or via co-transfection.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this invention in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., WO 95/13392); Burstein et al. (WO 98/23018); and Johnson et al, (U.S. Pat. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this invention.

### Compositions and Routes of Delivery

Any route of administration may be employed so long as that route and the amount administered are prophylactically or therapeutically useful.

In vivo administration of the components, e.g., delivered in a viral vector such as a lentivirus or AAV vector, and compositions containing them, can be accomplished by any suitable method and technique presently or prospectively known to those skilled in the art. The subject polynucleotides or polypeptides can be formulated in a physiologically- or pharmaceutically-acceptable form and administered by any suitable route known in the art including, for example, oral, nasal, rectal, transdermal, vaginal, and parenteral routes of administration. As used herein, the term parenteral includes subcutaneous, intradermal, intravenous, intramuscular, intraperitoneal, and intracisternal administration, such as by injection.

Administration of the compositions can be a single administration, or at continuous or distinct intervals as can be readily determined by a person skilled in the art. In one embodiment, a polynucleotide component is stably incorporated into the genome of a person of animal in need of treatment. Methods for providing gene therapy are well known in the art.

The compositions can also be administered utilizing liposome and nano-technology, slow release capsules, implantable pumps, and biodegradable containers, and orally or intestinally administered intact plant cells expressing the therapeutic product. These delivery methods can, advantageously, provide a uniform dosage over an extended period of time.

Suitable dose ranges for are generally about 10³ to 10¹⁵ infectious units of viral vector per microliter delivered in 1 to 3000 microliters of single injection volume. For instance, viral genomes or infectious units of vector per micro liter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector delivered in about 50, 100, 200, 500, 1000, or 2000 microliters. It should be understood that the aforementioned dosage is merely an exemplary dosage and those of skill in the art will understand that this dosage may be varied. Effective doses may be extrapolated from dose-responsive curves derived from in vitro or in vivo test systems.

In one embodiment, suitable dose ranges are generally about 10³ to 10¹⁵ infectious units of viral vector per microliter delivered in, for example, 1, 2, 5, 10, 25, 50, 75 or 100 or more milliliters, e.g.,1 to 10,000 milliliters or 0.5 to 15 milliliters, of single injection volume. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, or 10¹⁴ viral genomes or infectious units of viral vector. In one embodiment, suitable dose ranges, generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector, e.g., at least 1.2×10¹¹ genomes or infectious units, for instance at least 2×10¹¹ up to about 2×10¹² genomes or infectious units or about 1×10¹³ to about 5×10¹⁶ genomes or infectious units.

Administration of agents in accordance with the present invention can be achieved by direct injection of the composition or by the use of infusion pumps. For injection, the composition can be formulated in liquid solutions, e.g., in physiologically compatible buffers such as Hank's solution, Ringer's solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In one embodiment, the agent(s) may be administered by any route including parenterally. In one embodiment, the agent(s) may be administered by subcutaneous, intramuscular, or intravenous injection, orally, intrathecally, or intracranially, or by sustained release, e.g., using a subcutaneous implant. The the agent(s) may be dissolved or dispersed in a liquid carrier vehicle. For parenteral administration, the active material may be suitably admixed with an acceptable vehicle, e.g., of the vegetable oil variety such as peanut oil, cottonseed oil and the like. Other parenteral vehicles such as organic compositions using solketal, glycerol, formal, and aqueous parenteral formulations may also be used. For parenteral application by injection, the agent(s) may comprise an aqueous solution of a water soluble pharmaceutically acceptable salt of the active acids according to the invention, desirably in a concentration of 0.01-10%, and optionally also a stabilizing agent and/or buffer substances in aqueous solution. Dosage units of the solution may advantageously be enclosed in ampules.

The agent(s) may be in the form of an injectable unit dose. Examples of carriers or diluents usable for preparing such injectable doses include diluents such as water, ethyl alcohol, macrogol, propylene glycol, ethoxylated isostearyl alcohol, polyoxyisostearyl alcohol and polyoxyethylene sorbitan fatty acid esters, pH adjusting agents or buffers such as sodium citrate, sodium acetate and sodium phosphate, stabilizers such as sodium pyrosulfite, EDTA, thioglycolic acid and thiolactic acid, isotonic agents such as sodium chloride and glucose, local anesthetics such as procaine hydrochloride and lidocaine hydrochloride. Furthermore, usual solubilizing agents and analgesics may be added. Injections can be prepared by adding such carriers to the enzyme or other active, following procedures well known to those of skill in the art. A thorough discussion of pharmaceutically acceptable excipients is available in REMINGTON'S PHARMACEUTICAL SCIENCES (Mack Pub. Co., N.J. 1991). The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

When the agent(s) is administered in the form of a subcutaneous implant, the compound is suspended or dissolved in a slowly dispersed material known to those skilled in the art, or administered in a device which slowly releases the active material through the use of a constant driving force such as an osmotic pump. In such cases, administration over an extended period of time is possible.

The dosage at which the agent(s) is administered may vary within a wide range and will depend on various factors such as the severity of the disease, the age of the patient, etc., and may have to be individually adjusted. Compositions described herein may be employed in combination with another medicament. The compositions can appear in conventional forms, for example, aerosols, solutions, suspensions, or topical applications, or in lyophilized form.

Typical compositions include the agent(s) and a pharmaceutically acceptable excipient which can be a carrier or a diluent. For example, the active agent(s) may be mixed with a carrier, or diluted by a carrier, or enclosed within a carrier. When the active agent is mixed with a carrier, or when the carrier serves as a diluent, it can be solid, semi-solid, or liquid material that acts as a vehicle, excipient, or medium for the active agent. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active agent(s). Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer's solution, or an isotonic aqueous saline solution.

The agent(s) may be provided as a powder suitable for reconstitution with an appropriate solution as described above. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

Compositions contemplated by the present invention may include, for example, micelles or liposomes, or some other encapsulated form, or can be administered in an extended release form to provide a prolonged storage and/or delivery effect, e.g., using biodegradable polymers, e.g., polylactide-polyglycolide. Examples of other biodegradable polymers include poly(orthoesters) and poly(anhydrides).

Polymeric nanoparticles, e.g., comprised of a hydrophobic core of polylactic acid (PLA) and a hydrophilic shell of methoxy-poly(ethylene glycol) (MPEG), may have improved solubility and targeting to the CNS. Regional differences in targeting between the microemulsion and nanoparticle formulations may be due to differences in particle size.

Liposomes are very simple structures consisting of one or more lipid bilayers of amphiphilic lipids, i.e., phospholipids or cholesterol. The lipophilic moiety of the bilayers is turned towards each other and creates an inner hydrophobic environment in the membrane. Liposomes are suitable drug carriers for some lipophilic drugs which can be associated with the non-polar parts of lipid bilayers if they fit in size and geometry. The size of liposomes varies from 20 nm to few μm.

Mixed micelles are efficient detergent structures which are composed of bile salts, phospholipids, tri, di- and monoglycerides, fatty acids, free cholesterol and fat soluble micronutrients. As long-chain phospholipids are known to form bilayers when dispersed in water, the preferred phase of short chain analogues is the spherical micellar phase. A micellar solution is a thermodynamically stable system formed spontaneously in water and organic solvents. The interaction between micelles and hydrophobic/lipophilic drugs leads to the formation of mixed micelles (MM), often called swallen micelles, too. In the human body, they incorporate hydrophobic compounds with low aqueous solubility and act as a reservoir for products of digestion, e.g. monoglycerides.

Lipid microparticles includes lipid nano- and microspheres. Microspheres are generally defined as small spherical particles made of any material which are sized from about 0.2 to 100 μm. Smaller spheres below 200 nm are usually called nanospheres. Lipid microspheres are homogeneous oil/water microemulsions similar to commercially available fat emulsions, and are prepared by an intensive sonication procedure or high pressure emulsifying methods (grinding methods). The natural surfactant lecithin lowers the surface tension of the liquid, thus acting as an emulsifier to form a stable emulsion. The structure and composition of lipid nanospheres is similar to those of lipid microspheres, but with a smaller diameter.

Polymeric nanoparticles serve as carriers for a broad variety of ingredients. The active components may be either dissolved in the polymetric matrix or entrapped or adsorbed onto the particle surface. Polymers suitable for the preparation of organic nanoparticles include cellulose derivatives and polyesters such as poly(lactic acid), poly(glycolic acid) and their copolymer. Due to their small size, their large surface area/volume ratio and the possibility of functionalization of the interface, polymeric nanoparticles are ideal carrier and release systems. If the particle size is below 50 nm, they are no longer recognized as particles by many biological and also synthetic barrier layers, but act similar to molecularly disperse systems.

Thus, the composition of the invention can be formulated to provide quick, sustained, controlled, or delayed release, or any combination thereof, of the active agent after administration to the individual by employing procedures well known in the art. In one embodiment, the enzyme is in an isotonic or hypotonic solution. In one embodiment, for enzymes that are not water soluble, a lipid based delivery vehicle may be employed, e.g., a microemulsion such as that described in WO 2008/049588, the disclosure of which is incorporated by reference herein, or liposomes.

In one embodiment, the preparation can contain an agent, dissolved or suspended in a liquid carrier, such as an aqueous carrier, for aerosol application, The carrier can contain additives such as solubilizing agents, e.g., propylene glycol, surfactants, absorption enhancers such as lecithin (phosphatidylcholine) or cyclodextrin, or preservatives such as parabens.

### Exemplary Diseases

The composition(s) may be employed to prevent, inhibit or treat monogenic diseases including but not limited to lysosomal storage diseases, hemophilia, e.g., lack of or decreased factor VIII or IX production, sickle cell disease and thalassemia, e.g., lack of beta-globin or alpha-globin production. Lysosomal diseases and (parenthetically) related enzymes and proteins associated with diseases that are contemplated within the scope of the invention include, but are not limited to, Activator Deficiency/GM2 Gangliosidosis (beta-hexosaminidase), Alpha-mannosidosis (alpha-D-mannosidase), Aspartylglucosaminuria (aspartylglucosaminidase), Cholesteryl ester storage disease (lysosomal acid lipase), Chronic Hexosaminidase A Deficiency (hexosaminidase A), Cystinosis (cystinosis), Danon disease (LAMP2), Fabry disease (alpha-galactosidase A), Farber disease (ceramidase), Fucosidosis (alpha-L-fucosidase), Galactosialidosis (cathepsin A), Gaucher Disease (Type I, Type II, Type III) (beta-glucocerebrosidase), GM1 gangliosidosis (Infantile, Late infantile/Juvenile, Adult/Chronic) (beta-galactosidase), I-Cell disease/Mucolipidosis II (GlcNAc-phosphotransferase), Infantile Free Sialic Acid Storage Disease/ISSD (sialin), Juvenile Hexosaminidase A Deficiency ((hexosaminidase A), Krabbe disease (Infantile Onset, Late Onset) (galactocerebrosidase), Metachromatic Leukodystrophy (arylsulfatase A), Mucopolysaccharidoses disorders [Pseudo-Hurler polydystrophy/Muco lipidosis IIIA (N-acetylglucosamine-1-phosphotransferase), MPSI Hurler Syndrome (alpha-L iduronidase), MPSI Scheie Syndrome (alpha-L iduronidase), MPS I Hurler-Scheie Syndrome (alpha-L iduronidase), MPS II Hunter syndrome (iduronate-2-sulfatase), Sanfilippo syndrome Type A/MPS III A (heparan N-sulfatase), Sanfilippo syndrome Type B/MPS III B (N-acetyl-alpha-D-glucosaminidase), Sanfilippo syndrome Type C/MPS III C (acetyl-CoA, alpha-glucosaminide acetyltransferase, Sanfilippo syndrome Type D/MPS III D (N-acetylglucosamine-G-sulfate-sulfatase), Morquio Type A/MPS IVA (N-acetylgalatosamine-6-sulfate-sulfatase), Morquio Type B/MPS IVB (β-galactosidase-I), NAPS IX Hyaluronidase Deficiency (hyaluronidase), MPS VI Maroteaux-Lamy (arylsulfatase B), MPS VII Sly Syndrome (beta-glucuronidase), Mucolipidosis I/Sialidosis (alpha-N-acetyl neuraminidase), Mucolipidosis IIIC (N-acetylglucosamine-1-phosphotransferase), Mucolipidosis type IV (mucolipinl)], Multiple sulfatase deficiency (multiple sulfatase enzymes), Niemann-Pick Disease (Type A, Type B, Type C) (sphingomyelinase), Neuronal Ceroid Lipofuscinoses [(CLN6 disease—Atypical Late Infantile, Late Onset variant, Early Juvenile (ceroid-lipofuscinosis neuronal protein 6); Batten-Spielmeyer-Vogt/Juvenile NCL/CLN3 disease (battenin); Finnish Variant Late Infantile CLN5 (ceroid-lipofuscinosis neuronal protein 5); Jansky-Bielschowsky disease/Late infantile CLN2/TPP1 Disease (tripeptidyl peptidase 1); Kufs/Adult-onset NCL/CL-N4 disease; Northern Epilepsy/variant late infantile CLN8 (ceroid-lipofuscinosis neuronal protein 8); Santavuori-Haltia/Infantile CLN1/PPT disease (palmitoyl-protein thioesterase 1); Beta-mannosidosis (beta-mannosidase)], Tangier disease (ATP-binding cassette transporter ABCAI), Pompe disease/Glycogen storage disease type II (acid maltase), Pycnodysostosis (cathepsin K), Sandhoff disease/ Adult Onset/GM2 Gangliosidosis (beta-hexosaminidases A and B), Sandhoff disease/GM2 gangliosidosis—Infantile, Sandhoff disease/GM2 gangliosidosis—Juvenile (beta-hexosaminidases A and B), Schindler disease (alpha-N-acetylgalactosaminidas), Salla disease/Sialic Acid Storage Disease (sialin), Tay-Sachs/GM2 gangliosidosis (beta-hexosaminidase), and Wolman disease (lysosomal acid lipase), Sphingolipidosis, Hurmansky-Pudiak Syndrome (HPS1, HPS3, HPS4, HPS5, HPS6 and HPS7) Type 2—AP-3 complex subunit beta-1, Type 7—dysbindin), Chediak-Higashi Syndrome (lysosomal trafficking regulator protein), and Griscelli disease (Type 1: myosin-Va, Type 2: ras-related protein Rab-27A, Type 3: melanophilin).

Additional diseases (including related proteins) include the neurodegenerative diseases which include but are not limited to Parkinson's, Alzheimer's, Huntington's, and Amyotrophic Lateral Sclerosis ALS (superoxide dismutase), Hereditary emphysema (a 1-Antitrypsin), Oculocutaneus albinism (tyrosinase), Congenital sucrase-isomaltase deficiency (Sucrase-isomaltase), and Choroideremia (Repl) Lowe's Oculoceribro-renal syndrome (PIP2-5-phosphatase).

In one embodiment, the disorder or disease is Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease (Type I, Type II, Type III), GM1 gangliosidosis (Infantile, Late infantile/Juvenile, Adult/Chronic), I-Cell disease/Mucolipidosis II, Infantile Free Sialic Acid Storage Disease/ISSD, Juvenile Hexosaminidase A Deficiency, Krabbe disease (Infantile Onset, Late Onset), Metachromatic Leukodystrophy, Mucopolysaccharidoses disorders (Pseudo-Hurler polydystrophy/Mucolipidosis IIIA, MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, NAPS VII Sly Syndrome, Mucolipidosis I/Sialidosis, Mucolipidosis IIIC, Mucolipidosis type IV), Multiple sulfatase deficiency, Niemann-Pick Disease (Type A, Type B, Type C), Neuronal Ceroid Lipofuscinoses (CLN6 disease—Atypical Late Infantile, Late Onset variant, Early Juvenile; Batten-Spielmeyer-Vogt/Juvenile NCL/CLN3 disease; Finnish Variant Late Infantile CLN5; Jansky-Bielschowsky disease/Late infantile CLN2/TPP1 Disease; Kufs/Adult-onset NCL/CLN4 disease; Northern Epilepsy/variant late infantile CLN8; Santavuori-Haltia/Infantile CLN1/PPT disease; Beta-mannosidosis), Tangier disease, Pompe disease/Glycogen storage disease type II, Pycnodysostosis, Sandhoff disease/Adult Onset/GM2 Gangliosidosis, Sandhoff disease/GM2 gangliosidosis—Infantile, Sandhoff disease/GM2 gangliosidosis—Juvenile, Schindler disease, Salla disease/Sialic Acid Storage Disease, Tay-Sachs/GM2 gangliosidosis, Wolman disease, Sphingolipidosis, Hurmansky-Pudiak Syndrome, Chediak-Higashi Syndrome, or Griscelli disease.

### Genome Editing

Gene therapy holds promise for treating lysosomal diseases as it has potential for permanent, single-dose treatment. Currently, treatment protocols providing sustained therapeutic benefits with minimized safety risks for patients with lysosomal diseases are in desperate need. To this end, two constructs were designed: one encoding Cas9 targeting intron 1 of albumin locus, and the other encoding promoterless IDUA cDNA sequence. A total of four guide RNAs (gRNAs) were designed and transfected into fibroblast cells together with SaCas9. The ability of these gRNAs to guide Cas9-mediated cleavage at the albumin locus was evaluated via the Surveyor assay. Two days after hydrodynamic injection of these two plasmids into MPS I mice, only the mice receiving both plasmids (n=3) had significant higher IDUA enzyme activities in liver (2.7 fold of wildtype levels). Mice receiving the plasmid encoding promoterless cDNA donor (n=3) had no increase in IDUA activity. Deep sequencing showed that the % indels at the target locus was only 0.2%, which yielded substantial enzyme expression in 2 days. To further evaluate this strategy, the two constructs were packaged into AAV8 vectors, and were injected into neonatal MPS I mice at different doses. To determine the efficacy, IDUA, enzyme activities and GAG levels are measured, neurocognitive behaviors are assessed, and cellular vacuolation is evaluated by electron microscopy. Moreover, on-target and off-target gene modification rates, are assessed, residual Cas9 activity determined and vector copy number quantified. Results from this study are applicable for a clinical protocol of CRISPR-mediated in vivo genome editing to treat patients such as those with lysosomal storage disorders, mucoploysaccharidoses, e.g., MPS I patients, and blood disorders including hemophilia and thalassemia.

In a previous study with zinc finger nucleases (ZFNs), approximately 0.5% of mRNA from albumin locus was the fusion transcript, indicating a relatively low genome modification rate likely due to the use of 3 AAV vectors for transduction of a single hepatocyte. For humans, a higher dose may be needed and a higher dose brings about higher rates of off-target effects, more challenge for vector production and higher manufacturing costs. The CRISPR (Clustered Regulatory Interspaced Short Palindromic Repeats) system emerges as a powerful alternative because of its high targeting efficiency and ease of design. A new Cas9 ortholog, Staphylococcus aureus Cas9 (SaCas9), that is short enough to fit into AAV vectors, has been reported (Ran et al., 2015). In this study, no off-target events were observed in the mice after AAV delivery of SaCas9 and guide RNAs. More interestingly, three independent gene therapy studies using SaCas9 observed undetectable (Yang et al., 2016) or minimal (Nelson et al., 2016; Tabebordbar et al., 2016) off-target effects, indicating a very high specificity. Considering the high efficiency and specificity, a Cas based system, e.g., SaCas9, delivered by vectors including viral vectors, e.g., AAV vectors, was used. As opposed to 3 AAV vectors used in the study with ZFNs, this CRISPR/Cas system has 1 or 2 vectors. For the 2 vector system, in one embodiment, one vector encodes Cas9 and guide RNA, and the other encodes a promoterless donor sequence; in another embodiment, one vector encodes Cas9 and the other vector encodes the promoterless donor sequence and guide RNA. Assuming similar doses when using rAAV, and similar AAV transduction and nuclease targeting efficiency, the efficiency of successful genome editing by CRISPR is higher. Thus, such as CRISPR-mediated genome editing strategy may allow for the use of lower doses of AAV vectors for treating diseases including lysosomal diseases, which brings minimized risk, ease of vector production and less expense.

The design for CRISPR-mediated in vivo genome editing for MPS I mice includes, in one embodiment, i.v. administration of 2 different AAV vectors (AAV8 encoding Cas and gRNA, AAV8 carrying promoterless IDUA cDNA). With AAV carrying IDUA sequence and flanking homology sequences, IDUA sequence was inserted into albumin locus e through homology-directed repair (HDR). The splicing donor sequence at exon 1 of albumin locus interacted with the splicing acceptor preceding the donor sequence. Therefore, under control of the endogenous albumin promoter, a fusion transcript of albumin exon 1 and IDUA was generated. Since exon 1 of albumin mainly encodes signal peptide and was cleaved thereafter, the mature protein was IDUA enzyme only.

Cas9, e.g., SaCas9, and guide RNA can also mediate the insertion of HEXB cDNA into albumin locus and achieve expression of Hex enzyme. AAV8 vectors are liver-tropic, and SaCas9 is under control of a liver-specific promoter. By virtue of this, genome editing and transgene expression can be limited to hepatocytes. Systemic therapeutic benefits zfd achieved through a phenomenon called ‘cross correction’. A total of four guide RNAs (gRNAs) were designed and transfected into fibroblast cells together with SaCas9. The ability of these gRNAs to guide SaCas9-mediated cleavage at the albumin locus was evaluated via the SURVERYOR assay. The results showed that one of the gRNAs, g1 (5′GTATCTTTGATGACAATAATGGGGGAT3′; SEQ ID NO:3) mediated targeted DNA cleavage with the highest efficiency (11% indels, and was selected for future studies). Plasmids encoding SaCas9 and IDUA cDNA donor in MPS I mice through hydrodynamic injection. Only the mice receiving both plasmids had significant higher IDUA enzyme activities in liver (2.7 fold of wildtype levels). Mice receiving the plasmid encoding promoterless cDNA donor had no increase in IDUA activities, These results strongly support the feasibility of this CRISPR-mediated safe harbor genome editing strategy in treating MPS I mice.

### Neonatal or Prenatal Genome Editing

In order to establish a gene therapy protocol to achieve a satisfactory clinical outcome or good quality of life for patients with MPS I and other lysosomal diseases, a genome editing protocol which can provide sustained therapeutic benefits multiple tissues including the brain, and minimize the vector-associated risk was tested. A single administration of AAV vectors delivering the CRISPR system targeting, for example, the albumin locus of hepatocyte, may treat both systemic and neurological diseases of MPS I with minimized risks. The feasibility of this study is supported by preliminary data. As described herein, co-delivery of 2 AAV vectors, one of which a promoterless IDUA cDNA donor can efficiently facilitate insertion of IDUA sequence into the albumin locus through homology directed repair (HDR). The endogenous albumin promoter drives IDUA transgene expression, which is likely sufficient to treat both systemic and neurological diseases of MPS I through cross correction.

In one embodiment, the therapy is delivered to a neonate, e.g., a neonatal human. In one embodiment, the therapy is delivered to a fetus (prenatal delivery), e.g., a human fetus. For prenatal delivery, the vectors may be delivered via the maternal blood system, via a device such as a needle inserted into the uterus or the sacs associated therewith, e.g., the amniotic sac, into the fetal blood system (e.g., via the umbilical cord), into a fetal organ, e.g., lung or liver, or abdomen.

### Exemplary Genes for Use and Diseases to be Treated

Diseases that may be prevented, inhibited or treated using the methods disclosed herein include, but are not limited to, Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy (a deficiency of survivor of motor neuron-1, SMN-1), Spinocerebellar ataxia, Tangier disease, Tay-Sachs disease, Tuberous sclerosis, Von Hippel-Lindau syndrome, Williams syndrome, Wilson's disease, or Zellweger syndrome. In one embodiment, the disease is a lysosomal storage disease, e.g., a lack or deficiency in a lysosomal storage enzyme. Lysosomal storage diseases include, but are not limited to, mucopolysaccharidosis (MPS) diseases, for instance, mucopolysaccharidosis type I, e.g., Hurler syndrome and the variants Scheie syndrome and Hurler-Scheie syndrome (a deficiency in alpha-L-iduronidase); Hunter syndrome (a deficiency of iduronate-2-sulfatase); mucopolysaccharidosis type III, e.g., Sanfilippo syndrome (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV, e.g., Morquio syndrome (a deficiency of galactosamine-6-sulfate sulfatase or beta-galactosidase); mucopolysaccharidosis type VI, e.g., Maroteaux-Lamy syndrome (a deficiency of arylsulfatase B); mucopolysaccharidosis type II; mucopolysaccharidosis type III (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV (A or B; a deficiency of galactosamine-6-sulfatase and beta-galatacosidase); mucopolysaccharidosis type VI (a deficiency of arylsulfatase B); mucopolysaccharidosis type VII (a deficiency in beta-glucuronidase); mucopolysaccharidosis type VIII (a deficiency of glucosamine-6-sulfate sulfatase), mucopolysaccharidosis type IX (a deficiency of hyaluronidase); Tay-Sachs disease (a deficiency in alpha subunit of beta-hexosaminidase); Sandhoff disease (a deficiency in both alpha and beta subunit of beta-hexosaminidase); GM1 gangliosidosis (type I or type II); Fabry disease (a deficiency in alpha galactosidase); metachromatic leukodystrophy (a deficiency of aryl sulfatase A); Pompe disease (a deficiency of acid maltase); fucosidosis (a deficiency of fucosidase); alpha-mannosidosis (a deficiency of alpha-mannosidase); beta-mannosidosis (a deficiency of beta-mannosidase), neuronal ceroid lipofuscinosis (NCL) (a deficiency of ceroid lipofucinoses (CLNs), e.g., Batten disease having a deficiency in the gene product of one or more of CLN1 to CLN14), and Gaucher disease (types I, II and III; a deficiency in glucocerebrosidase), as well as disorders such as Hermansky-Pudlak syndrome; Amaurotic idiocy; Tangier disease; aspartylglucosaminuria; congenital disorder of glycosylation, type Ia; Chediak-Higashi syndrome; macular dystrophy, corneal, 1; cystinosis, nephropathic; Fanconi-Bickel syndrome; Farber lipogranulomatosis; fibromatosis; geleophysic dysplasia; glycogen storage disease I; glycogen storage disease Ib; glycogen storage disease Ic; glycogen storage disease III; glycogen storage disease IV; glycogen storage disease V; glycogen storage disease VI; glycogen storage disease VII; glycogen storage disease 0; immunoosseous dysplasia, Schimke type; lipidosis; lipase b; mucolipidosis II; mucolipidosis II, including the variant form; mucolipidosis IV; neuraminidase deficiency with beta-galactosidase deficiency; mucolipidosis I; Niemann-Pick disease (a deficiency of sphingomyelinase); Niemann-Pick disease without sphingomyelinase deficiency (a deficiency of a npc1 gene encoding a cholesterol metabolizing enzyme); Refsum disease; Sea-blue histiocyte disease; infantile sialic acid storage disorder; sialuria; multiple sulfatase deficiency; triglyceride storage disease with impaired long-chain fatty acid oxidation; Winchester disease; Wolman disease (a deficiency of cholesterol ester hydrolase); Deoxyribonuclease I-like 1 disorder; arylsulfatase E disorder; ATPase, H+ transporting, lysosomal, subunit 1 disorder; glycogen storage disease IIb; Ras-associated protein rab9 disorder; chondrodysplasia punctata 1, X-linked recessive disorder; glycogen storage disease VIII; lysosome-associated membrane protein 2 disorder; Menkes syndrome; congenital disorder of glycosylation, type Ic; and sialuria. Replacement of less than 20%, e.g., less than 10% or about 1% to 5% levels of lysosomal storage enzyme found in nondiseased mammals, may prevent, inhibit or treat neurological symptoms such as neurological degeneration in mammals. In one embodiment, the disease to be prevented, inhibited or treated with a particular gene includes, but is not limited to, MPS I (IDUA), MPS II (IDS), MPS IIIA (Heparan-N-sulfatase; sulfaminidase), MPS IIIB (alpha-N-acetyl-glucosaminidase), MPS IIIC (Acetyl-CoA:alpha-N-acetyl-glucosaminide acetyltransferase), MPS IIID (N-acetylglucosamine 6-sulfatase), MPS VII (beta-glucoronidase), Gaucher (acid beta-glucosidase), Alpha-mannosidosis (alpha-mannosidase), Beta-mannosidosis (beta-mannosidase) Alpha-fucosidosis (alpha-fucosidase), Sialidosis (alpha-sialidase), Galactosialidosis (Cathepsin A), Aspartylglucosaminuria (aspartylglucosaminidase), GM1-gangliosidosis (beta-galactosidase), Tay-Sachs (beta-hexosaminidase subunit alpha), Sandhoff (beta-hexosaminidase subunit beta), GM2-gangliosidosis/variant AB (GM2 activator protein), Krabbe (galactocerebrosidase), Metachromatic leukodystrophy (arylsulfatase A), and other neurologic disorders including but not limited to Alzheimer's disease (expression of an antibody, such as an antibody to beta-amyloid, or an enzyme that attacks the plaques and fibrils associated with Alzheimer's), or Alzheimer's and Parkinson's diseases (expression of neuroprotective proteins including but not limited to GDNF or Neurturin).

Below is an alignment of the sequences of Hex A (SEQ ID NO:23) and HexM (alpha subunit is a human sequence; the mu sequence was formed by introducing some areas of the beta subunit into the alpha subunit), and a synthetic Hex (homodimer of the mu subunit) (SEQ ID NO:24).

| | | | |
|---|---|---|---|
| α-subunit | 1 | MTSSRLWFSLLLAAAFAGRATALWPWPQMFQTSDQRYVLYPNNFQFQYDVSSAAQPGCSV | 60 |
| μ-subunit | 1 | MTSSRLWFSLLLAAAFAGRATALWPWPQNPQTSDQRYVLYPNNFQFQYDVSSAAQPGCSV | 60 |
| α-subunit | 61 | LDEAFQRYRDLLFGSGSWPRPYLTGKRHTLEKNVLVVSVVTPGCNQLPTLESVENYTLTI | 120 |
| μ-subunit | 61 | LDEAFQRYRDLLFGSGSWPRPYLTGKRHTLEKNVLVVSVVTPGCNQLPTLFSVENYTLTI | 120 |
| α-subunit | 121 | NDDQCLLLSETVWGALRGLETFSQLVWKSAEGTFFINKTEIEDFPRFPHRGLLLDTSRHY | 180 |
| μ-subunit | 121 | NDDQCLLLSETVWGALRGLETFSQLVWKSAEGTFFINKTEIEDFPRFPHRGLLLDTSRGY | 180 |
| α-subunit μ-subunit | 181 181 | 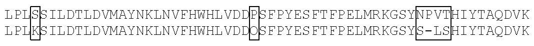 | 240 239 |
| α-subunit | 241 | EVIEYARLRGIRVLAEFDTPGHTLSWGPGIPGLLTPCYSGSEPSGTFGPVNPSLNNTYEF | 300 |
| μ-subunit | 240 | EVIEYARLRGIRVLAEFDTPGHTLSWGPGIPGLLTPCYSGSEPSGTFGPVNPSLNNTYEF | 299 |
| α-subunit | 301 | MSTFFLEVSSVFPDFYLHLGGDEVDFTCWKSNPEIQDFMRKKGRGEDFKQLESFYIQTLL | 360 |
| μ-subunit | 300 | MSTFFLEVSSVFPDFYLHLGGDEVDFTCWKSNPEIQDFMRKKGFGEDFKQLESFYIQTLL | 359 |
| α-subunit | 361 | DIVSSYGKGYVVWQEVFDNKVKIQPDTIIQVWREDIPVNYMKELELVTKAGFRALLSAPW | 420 |
| μ-subunit | 360 | DIVSSYGKGYVVWQEVFDNKVKIQPDTIIQVWREDPPVNYMKELELVTKAGFRALLSAPW | 419 |
| α-subunit μ-subunit | 421 420 | 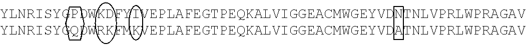 | 480 479 |
| α-subunit μ-subunit | 481 480 | 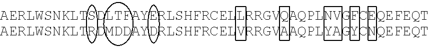 | 529 528 |

Met Thr Ser Ser Arg Leu Trp Phe Ser Leu Leu Leu Ala Ala Ala Phe Ala Gly Arg Ala Thr Ala Leu Trp Pro Trp
Pro Gln Asn Phe Gln Thr Ser Asp Gln Arg Tyr Val Leu Tyr Pro Asn Asn Phe Gln Phe Gln Tyr Asp Val Ser Ser
Ala Ala Gin Pro Gly Cys Ser Val Leu Asp Glu Ala Phe Gin Arg Tyr Arg Asp Leu Leu Phe Gly Ser Gly Ser Trp
Pro Arg Pro Tyr Leu Thr Gly Lys Arg His Thr Leu Glu Lys Asn Val Leu Val Val Ser Val Val Thr Pro Gly Cys
Asn
Gin Leu Pro Thr Leu Glu Ser Val Glu Asn Tyr Thr Leu Thr Ile Asn Asp Asp Gln Cys Leu Leu Leu Ser Glu Thr
Val Trp Gly Ala Leu Arg Gly Leu Glu Thr Phe Ser Gln Leu Val Trp Lys Ser Ala Glu Gly Thr Phe Phe Ile Asn
Lys
Thr Glu Ile Glu Asp Phe Pro Arg Phe Pro His Arg Gly Leu Leu Leu Asp Thr Ser Arg His Tyr Leu Pro Leu Lys
Ser Ile Leu Asp Thr Leu Asp Val Met Ala Tyr Asn Lys Leu Asn Val Phe His Trp His Leu Val Asp Asp Gin Ser
Phe Pro Tyr Glu Ser Phe Thr Phe Pro Glu Leu Met Arg Lys Gly Ser Tyr Ser Leu Ser His Ile Tyr Thr Ala Gln
Asp Val Lys Glu Val Ile Glu Tyr Ala Arg Leu Arg Gly Ile Arg Val Leu Ala Glu Phe Asp Thr Pro Gly His Thr
Leu
Ser Trp Gly Pro Gly Ile Pro Gly Leu Leu Thr Pro Cys Tyr Ser Gly Ser Glu Pro Ser Gly Thr Phe Gly Pro Val
Asn
Pro Ser Leu Asn Asn Thr Tyr Glu Phe Met Ser Thr Phe Phe Leu Glu Val Ser Ser Val Phe Pro Asp Phe Tyr Leu
His Leu Gly Gly Asp Glu Val Asp Phe Thr Cys Trp Lys Ser Asn Pro Glu Ile GIn Asp Phe Met Arg Lys Lys Gly
Phe Gly Glu Asp Phe Lys Gln Leu Glu Ser Phe Tyr Ile Gin Thr Leu Leu Asp Ile Val Ser Ser Tyr Gly Lys Gly
Tyr
Val Val Trp Gln Glu Val Phe Asp Asn Lys Val Lys Ile Gln Pro Asp Thr Ile Ile Gin Val Trp Arg Glu Asp Ile
Pro
Val Asn Tyr Met Lys Glu Leu Glu Leu Val Thr Lys Ala Gly Phe Arg Ala Leu Leu Ser Ala Pro Trp Tyr Leu Asn
Arg Ile Ser Tyr Gly Gln Asp Trp Arg Lys Phe Tyr Lys Val Glu Pro Leu Ala Phe Glu Gly Thr Pro Glu Gln Lys
Ala
Leu Val Ile Gly Gly Glu Ala Cys Met Trp Gly Glu Tyr Val Asp Ala Thr Asn Leu Val Pro Arg Leu Trp Pro Arg
Ala
Gly Ala Val Ala Glu Arg Leu Trp Ser Asn Lys Leu Thr Arg Asp Met Asp Asp Ala Tyr Asp Arg Leu Ser His Phe
Arg Cys Glu Leu Val Arg Arg Gly Val Ala Ala Gin Pro Leu Tyr Ala Gly Tyr Cys Asn Gin Glu Phe Glu Gln Thr
(SEQ ID NO: 25) (WO 2015/150922, the disclosure of which is incorporated by reference herein)

### Exemplary Disease to be Treated: MPS I Disease

Mucopolysaccharidosis type I (MPS I) has an incidence of approximately 1 out of 100,000 births and results from mutations in the gene encoding the lysosomal enzyme α-L-iduronidase (IDUA) (Neufeld & Muenzer, 2001). Deficiency of IDUA gives rise to progressive lysosomal accumulation of glycosaminoglycans (GAG) heparan and dermatan sulfate. The signs and symptoms of MPS I may become manifest in childhood, or later in life. Based on the first appearance of these features and the severity, MPS I disorders are differentiated into three subtypes, from severe infantile ‘Hurler syndrome’ (MPS IH) to intermediate Hurler-Scheie syndrome (MPS IHS) to attenuated Scheie syndrome (MPS IS) (Neufeld & Muenzer, 2001). Patients with Scheie or Hurler-Scheie diseases have symptoms including growth delay and short stature, progressive life-threatening aortic and intracardiac valvular disease, skeletal dysplasias with deformities contractures, carpal tunnel syndrome, spinal cord compression, corneal opacification all of which lead to severe disability and early demise (Neufeld & Muenzer, 2001). These features appear much earlier infancy for those with null mutations for which the term Hurler syndrome applies; hepatosplenomegaly, dysmorphic facial features, hydrocephalus, mental retardation, and neurodegeneration are prominent. Without treatment, children with Hurler syndrome uniformly die between 5-10 years of age. Current treatments only mitigate some of the serious and life-threatening medical problems; survivors may live longer but with progressive and intractable disabilities, and a very poor quality of life. Even the best expectations for patients receiving multiple ‘combined therapies’ face a life of multiple serious worsening disabilities, ongoing dependency on families and the expensive medical care system. Many are not employable, or go on to disability early in life.

Hematopoietic stem cell transplantation (HSCT) was proposed as a systemic therapy (Hobbs et al., 1981) and subsequently found to halt or reverse some somatic features, prevent neurodegenerative disease including reversal of hydrocephalus (reduction of lumbar spinal fluid opening pressure) and stabilize developmental quotient, and thus showed unexpected metabolic correction across the blood-brain barrier (Whitley et al., 1986). Subsequent studies have found that FDA-approved weekly intravenous enzyme replacement therapy (ERT, laronidase, Aldurazyme®) mitigates the progression of some somatic disease. However, at FDA-approved dosing (0.58 mg/kg weekly) does not provide metabolic correction in the central nervous system (CNS); IV laronidase (Aldurazyme®) does not prevent progressive mental retardation in children. Further, although HSCT has been shown superior to ERT monotherapy in MPS I, the suboptimal long-term outcomes of ERT monotherapy must be considered in the context of access issues to HSCT worldwide (Eisengart et al, 2018). Combination therapy—ERT pre and/or post successful HSCT—has also proven less than efficacious due to the development of treatment negating anti-IDUA antibodies (Xue et al., 2016). There remains a clear need for better, more accessible, and less expensive therapy for both the CNS and somatic disease burden in MPS I.

This tantalizing history of treatments for Hurler syndrome—and desperate need for a safer and effective systemic therapy that treats the CNS—make Hurler syndrome a target disease to test the PS system, a more efficient platform for gene editing we have developed for the treatment of lysosomal diseases. The recent federal mandate to implement newborn screening for Hurler syndrome (now active in 8 states, and increasing across the nation) removes barriers to patient recruitment and treatment of subjects (n=6) immediately after birth. Of note, HSCT for Hurler syndrome, generally deferred until 6 months of age) provides a ‘rescue’ procedure, if needed.

### Advantages of the PS system

Both HSCT and ERT are available treatments for MPS I. However, these treatments have significant limitations. HSCT can lead to prolonged survival (Hobbs et al., 1981), somatic improvements, and partial neurological benefits, but the procedure is associated with morbidity or mortality (Whitley et al., 1986; Eisengart et al., 2018). ERT is of limited use due to the need for frequent and life-long administration, high cost (>$200,000 annually), and negligible neurological benefits (Wraith et al., 2004). Compared with weekly ERT, the PS gene editing system can provide a magnitude higher enzyme with a single administration. For a 75-kg patient with MPS I, the weekly ERT dose is approximately 43.5 mg, and ERT half-life is 1.5 to 3.6 hours. The PS system uses CRISPR/Cas9 gene editing for MPS I. The PS system inserts a therapeutic transgene in the albumin intron 1 locus, and then therapeutic proteins are expressed under the control of the endogenous albumin promoter. The albumin promoter is very highly expressed. Normal albumin levels in the blood are 40-50 mg/mL and synthesized from the liver at a rate of 105 g/week. Based on an estimation, only 0.05% of albumin production to provides the same amount of IDUA enzyme provided by ERT. In a preliminary study using the PS gene editing system, a 50-fold higher efficiency was achieved than a ZEN study. Therefore, the PS system to provide a magnitude higher enzyme than ERT with a single intravenous administration. Further, pulsatile ERT may trigger drug-neutralizing antibodies. This would be obviated by continuous delivery of enzyme by PS gene editing. Such continuous enzyme delivery has been used to create immune tolerance from ERT, and eliminate neutralizing antibodies against lysosomal enzymes. Thus, the PS system may achieve better safety and efficacy than ERT. Moreover, preliminary data showed that the PS system achieved significant neurological benefits in animal models of lysosomal diseases, which is another critical advantage over ERT.

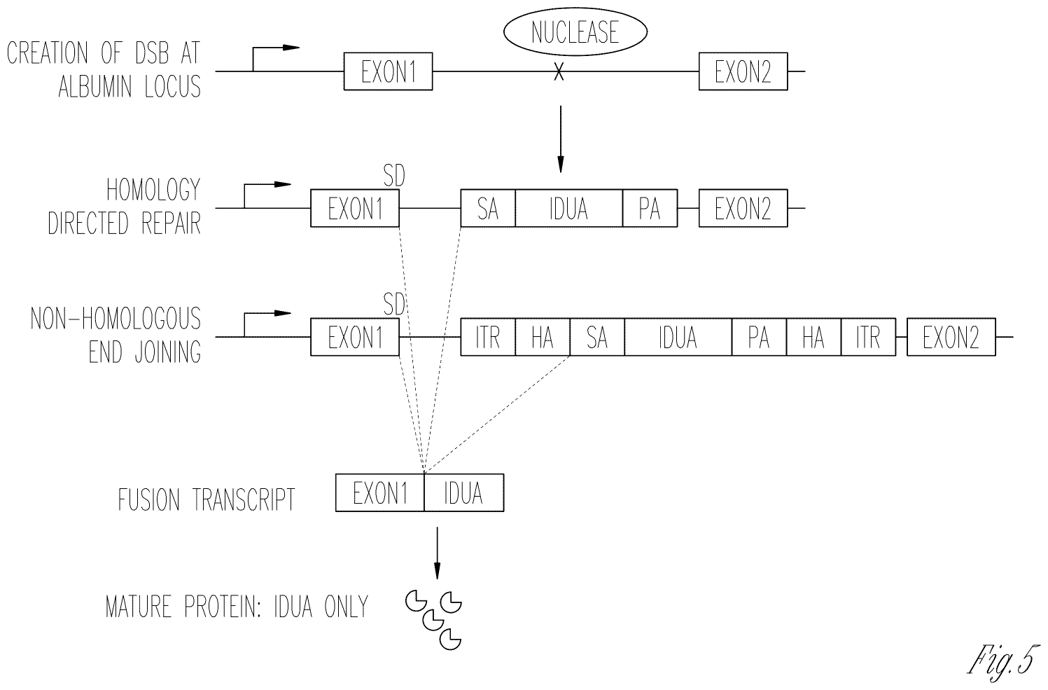

Most patients suffering from lysosomal diseases are children, and normal growth requires continuous cell divisions. AAV gene therapy faces this major problem of vector dilution, a significant issue that most investigators are ignoring, but will become a major problem as children grow and mature. As shown in clinical trials of AAV gene therapy for hemophilia B, the transgene expression in humans reduces over time. This could be due to the non-integrating nature of the AAV vector. It was shown that transgene expression from episomal AAV vectors was rapidly lost after one round of cell division (Kishnani et al., 2016), leading to a gradual decline of therapeutic effects (FIG. 4). Unfortunately, secondary administration of AAV vectors often fails to rescue expression, due to the immune response to primary vector delivery. Therefore, the major advantage of the PS system over traditional AAV gene therapy is its ability to create life-long enzyme replacement therapy, overcoming the issue of vector dilution, and will provide ongoing efficacy after the first few years following treatment.

The AAV in the clinical trial for MPS I is administered through direct injection into the brain, which may have several drawbacks: 1) highly invasive administration; 2) difficulty in achieving uniform and global distribution throughout the brain (Passini et al., 2002); 3) the inability to treat systemic diseases that become prominent when lifespan is extended because neurological diseases are treated (Cachón-González & Wang, 2012); and 4) genotoxicity due to overexpression of lysosomal enzyme in neurons (Golebiowski et al., 2017). In one embodiment, the use of the PS gene editing system results in a liver-targeting approach, e.g., through intravenous administration. In one embodiment, a therapeutic strategy is one that delivers enzyme uniformly to the brain. Considering that each neuron is estimated to be approximately 15 μm from blood vessels, the bloodstream is an ideal conduit for enzyme delivery. In addition, targeting the liver has additional advantages: 1) substantial clinical experience with liver-targeting gene therapy; and 2) hepatic transgene expression known to induce immune tolerance (Finn et al., 2010; Mingozzi et al., 2003).

Gene editing surmounts barriers to clinical relevance encountered by other methods as it enables long-term transgene expression and minimizes insertional mutagenesis risk from random integration. Furthermore, the PS system has a magnitude higher expression than other gene editing systems. As shown in clinical trials for treating MPS VI (Brunetti-Pierri, 2019) and hemophilia B (Doshi & Arruda, 2018), relatively low efficiency is a major obstacle for gene therapy. Increasing the dose will bring about a higher risk of toxicity, more challenging vector production, and increased manufacturing costs. The PS system only requires two vectors: one AAV vector encoding Cas9 and guide RNA, and the other encoding a promoterless donor sequence. Assuming similar doses, AAV transduction and nuclease targeting efficiency, the efficiency of successful genome editing by the PS system is expected to be higher. Preliminary data showed that the PS system showed 50-fold higher efficiency than a ZFN study (25 fold of IDUA enzyme activity with <50% of the dose). Therefore, an advantage of the PS system over the ZFN approach is its magnitude higher efficiency to achieve sufficient therapeutic benefits in human patients.

### Target Product Profile

A target product profile was developed that shows the goals of the test article, including disease indication and stage, treatment duration, delivery mode, dosing regimen, patient population, and standards for clinical efficacy (Table 1).

TABLE 1
Target product profile of PS822.

| Product Targets | Minimum Acceptable Result | Ideal Results |
|---|---|---|
| Primary Product Indication | mucopolysaccharidosis type | mucopolysaccharidosis type |
| | I as an adjunct therapy | I as a sole therapy |
| Patient Population | Selected affected individuals | All affected individuals |
| Treatment Duration | Lifetime | Lifetime from birth |
| Delivery Mode | IV | IV |
| Dosage Form | Frozen individual vials of 2 | Frozen individual vials of 2 |
| | different vectors that thawed | different vectors that are |
| | for co-administration | thawed for co-administration |
| Regimen | Single infusion over up to 24 | Single infusion 2-4 hours |
| | hours | |
| Efficacy | Diminished progression of | Prevention of all disease |
| | some disease features of | features of Hurler syndrome, |
| | mucopolysaccharidosis type I | including neurodegenerative |
| | | disease and bone dysplasia |
| | | when given at birth or earlier |
| Risk/Side Effect | Treatable, transient infusion | No adverse infusion reactions |
| | reactions at the time of | or other adverse effects of the |
| | infusion and without long- | treatment |
| | term negative impact on | |
| | quality of life or longevity | |
| Therapeutic Modality | In vivo gene editing | In vivo gene editing |

Improvements in motor function were chosen as the minimum acceptable result based on benefits provided by the standard of care. Neurological improvements were added as an ideal result based on the medical need in MPS I and preliminary results in MPS I mice. For the patient population, the Hurler subtype of MPS I was chosen based on the mutation in MPS I mice models. Efficacy parameters for the minimum acceptable results and ideal results were based on clinical trials with laronidase (Aldurazyme®), the ERT for MPS I. However, improvements in Bayley and Wechsler testing capture neuropsychological improvements would be expected from the PS system (Shapiro et al, 2017).

### Clinical Study

The data generated are used for initiating a Phase I/II clinical trial. Herein the Phase I/II clinical trial protocol and the feasibility are described. The Phase I/II clinical trial enrolls 6 infants (ages birth to 2 years of age) who have the three key diagnostic criteria of (1) deficient IDUA enzyme activity, (2) abnormally increased urine GAG, and (3) IDUA mutations consistent with the ‘severe’ phenotype of Hurler syndrome. Specific enrollment criteria otherwise match standard general criteria for this type of study, i.e., fully informed consent, lack of co-morbidities or other factors that would prevent completion of the clinical trial, etc. Subjects would be treated on the University of Minnesota Bone Marrow Transplant Intensive Care Unit. The infusion procedure includes actual hospitalization for only a brief, few-day observation period.

Subjects undergo a standard pre- and post-treatment evaluation as has been done for more than 30 years beginning with the initial transplant procedure of Hurler syndrome in the USA (Sep. 13, 1983). This includes physical examination, radiographic studies, routine blood (safety) tests, and also (under anesthesia) MRI of brain and abdomen for volumetrics, lumbar spinal tap (for opening pressure and CSF biomarkers). As an outpatient, subjects have extensive age-appropriate psychometric testing, ENT, Ophthalmology, and other specialty evaluations. This systematic testing is done prior to treatment and then at 6-month intervals for the duration of the study which will be 36 months after treatment.

### Data

### Scientific Rationale

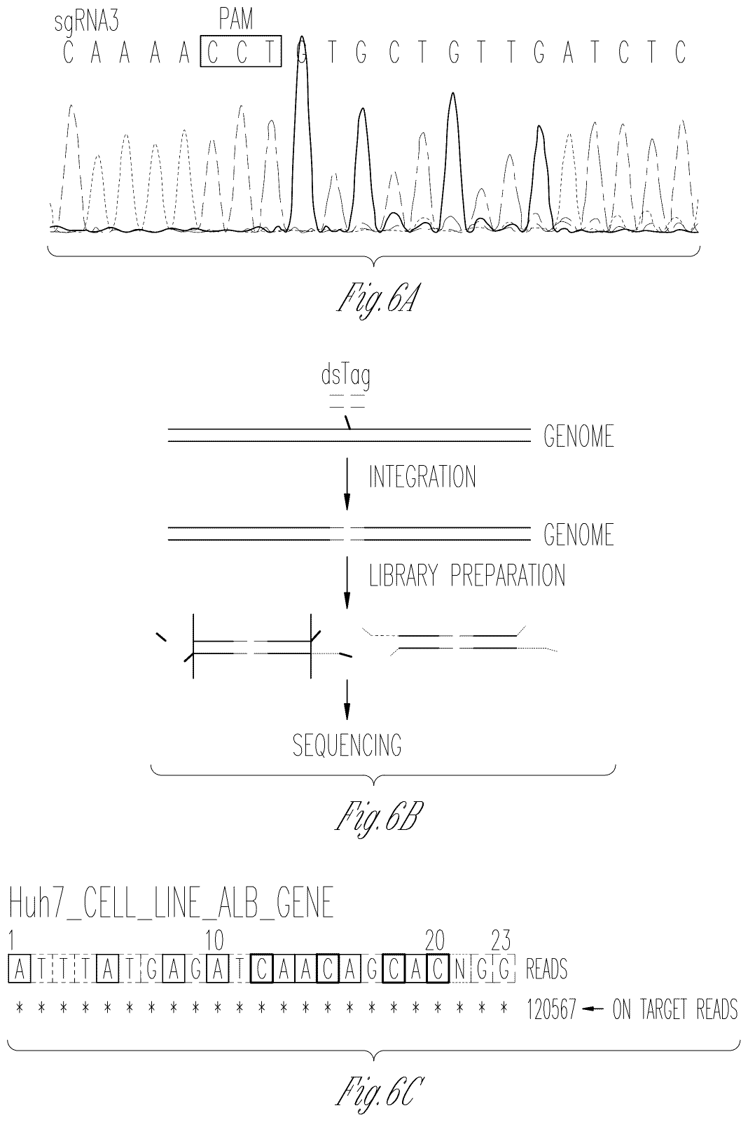

In 2018, the FDA approved an IND application based on an in vivo delivery of the CRISPR/Cas9 system for treating Leber Congenital Amaurosis type 10 (Maeder et al., 2019), which supports the use of CRISPR/Cas9 in patients. More recently, a study used a GOTI method (genome-wide off-target analysis by two-cell embryo injection) to determine off-target effects by editing a blastomere of two-cell mouse embryos using Cas9 (Zuo et al., 2019). This method separates off-target signals from background noise by using cells with an identical genetic background as controls. Comparison between the whole genome of progeny cells of edited vs non-edited blastomeres identified very rare off-target events of Cas9 (similar to spontaneous mutations). These facts support the safety and potential clinical application of CRISPR/Cas9. Therefore, a PS gene editing system was designed utilizing CRISPR/Cas9. The design for the PS system is shown in FIG. 5. Cas9 and gRNA mediate the insertion of a therapeutic transgene (promoterless cDNA) into the albumin intron 1 locus of hepatocytes. This insertion utilizes both HDR and NHEJ mechanisms, and thus the PS system can work in both dividing and non-dividing cells. A therapeutic transgene is expressed under the control of a highly expressed promoter, e.g., the endogenous albumin promoter. Systemic therapeutic benefits are achieved through cross correction. Data in MPS I mice showed that the PS system achieved a 50-fold higher efficiency than that achieved by the ZEN system. Therefore, PS gene editing enables usage of lower doses of AAV vector to treat lysosomal diseases, which minimizes risk, eases vector production, and is less costly.

Multiple preclinical studies with high doses of ERT that are relatively high compared to usual doses of ERT can be used to treat patients (Table 2). One outcome of treatment with the PS system is to effectively treat neurological complications of MPS, e.g., MPS I, through cross correction.

These studies showed that a high level of enzyme in circulation could facilitate entry of enzyme into the brain. Possible mechanisms may include: 1) impaired integrity of BBB due to disease; 2) fluid-phase pinocytosis; 3) extracellular pathway; 4) residual mannose 6-phosphate receptor (M6PR) or other uncharacterized receptors. Moreover, preliminary data in MPS I and Sandhoff mice showed that the PS gene editing system achieved significant neurological benefits (significant enzyme activities and substrate reduction in the brain, as well as improved neurobehaviors), lending further strength to this.

TABLE 2
Previous preclinical studies show that high doses of ERT can treat neurological complications.

| | Dose | Clinical dose | Brain enzyme | Brain storage | |
|---|---|---|---|---|---|
| Mouse model | (mg/kg) | (mg/kg) | activity (% Normal) | reduced (% Affected) | References |
| α-mannosidosis | 18.3 | 1 | N/A | 74% | 51 |
| α-mannosidosis | 36.6 | 1 | 15% | 50% | 52 |
| Metachromatic leukodystrophy | 20 | N/A | N/A | 30% | 53 |
| Metachromatic leukodystrophy | 50 | N/A | N/A | 34% | 54 |
| Aspartylglycosaminuria | 10 | N/A | 10% | 20% | 55 |
| Krabbe disease | 6 | N/A | 7% | 18% | 56 |
| MPS II | 10 | 0.5 | 5% | N/A | 57 |
| MPS IIIA | 20 | N/A | 22% | 0 | 58 |
| MPS VII | 20 | 2 | 2.50% | N/A | 59 |
| MPS I | 20 | 0.58 | 97% | 63% | 60 |

### Product

Mouse surrogate PS822 reagents First, mouse surrogate PS822 reagents were designed to target the mouse albumin intron 1 locus, and tested in vitro and in vivo. The mouse surrogate PS822 is supplied as two individually packaged recombinant AAV2/8 vectors. It includes two components: AAV2/8-SaCas9-sgRNA and AAV2/8-hIDUA.

In the first vector, SaCas9 expression is under the control of the liver-specific promoter: tyrosine hormone-binding globulin (TBG) promoter, The TBG promoter is specifically and highly active in hepatocytes, the intended target tissue, but is inactive in non-liver cell and tissue types; this prevents Cas9 expression and activity in non-target tissues. The polyA sequences are derived from the bovine growth hormone gene. In addition, a U6 promoter and a sgRNA sequence are included to direct the Cas9 cleavage activity at the target locus.

The second vector encodes the promoterless human IDUA sequence (the signal peptide sequence removed). A splice acceptor (SA) sequence, upstream of the IDUA donor sequence, is present to allow efficient splicing of hIDUA transgene into the mature mRNA from the albumin locus and is effective with both types of the donor integration mechanisms NHEJ or HDR²³. Sequences homologous to the cleavage site at the human albumin intron 1 locus are designed to flank the hIDUA transgene. The arms of homology are present to facilitate targeted integration of the hIDUA transgene at the albumin intron 1 locus via HDR.

Human PS822 reagents PS822 targets the human albumin intron 1 locus. The human PS822 is also supplied as two individually packaged recombinant AAV2/6 vectors for IV administration. Compared with the mouse PS822 surrogate reagents, five specific changes were introduced in an exemplary human PS822 test article (Table 3).

TABLE 3
Changes made in the human PS822 test article compared with mouse surrogate reagents.

1) AAV2/6 vectors instead of AAV2/8 vectors are used for better liver tropism in human;
2) Redesigned homology arms for targeting the human albumin intron 1 locus;
3) Cas9 and sgRNA sequences are separated into 2 vectors to minimize the continuous cutting risk;
4) SpCas9 instead of SaCas9 is used for more efficient cleavage at the target locus;
5) Introduction of 9 mutations in the homology arms where is homologous to the target locus to
minimize the continuous cutting risk;

First. AAV2/8 vectors are used in the mouse studies. but AAV2/6 vectors are used in the clinical trials for its better liver tropism in human. Second, the target locus in human albumin intron 1 locus is different than the one in the mouse albumin intron 1 locus. Therefore, the homology arms are redesigned for mediating efficient HDR at the target locus. Third, Cas9 and sgRNA sequences are separated into 2 vectors to minimize the continuous cutting risk. The human PS822 reagents include 2 components: AAV2/6-SpCas9 and AAV2/6-hIDUA-sgRNA. Since Cas9 nuclease cannot cleave the DNA without the gRNA, this arrangement can reduce the possibility of continuous cutting. Fourth, the human PS822 reagents include SpCas9 instead of SaCas9 because SpCas9 showed remarkably more efficient cleavage at the target locus when tested in human hepatocytes. Previous studies showed the feasibility of fitting SpCas9 into AAV vectors (Liao et al., 2017; Nishiyama et al., 2017). Fifth, to reduce the risk of continuous cutting by Cas9 at the target locus, mutations were introduced in the homology arms where they were homologous to the target locus and PAM site. Therefore, after one round of gene editing, the target locus is changed and would not be recognized by Cas9 anymore. Mutating regions of HA that are homologous to the target locus has been shown to successfully avoid continuous cutting (Ohmori et al., 2017; Okamoto et al., 2019). With all these changes, the human PS822 reagents are likely to be superior to the mouse PS822 reagents in safety and efficacy.

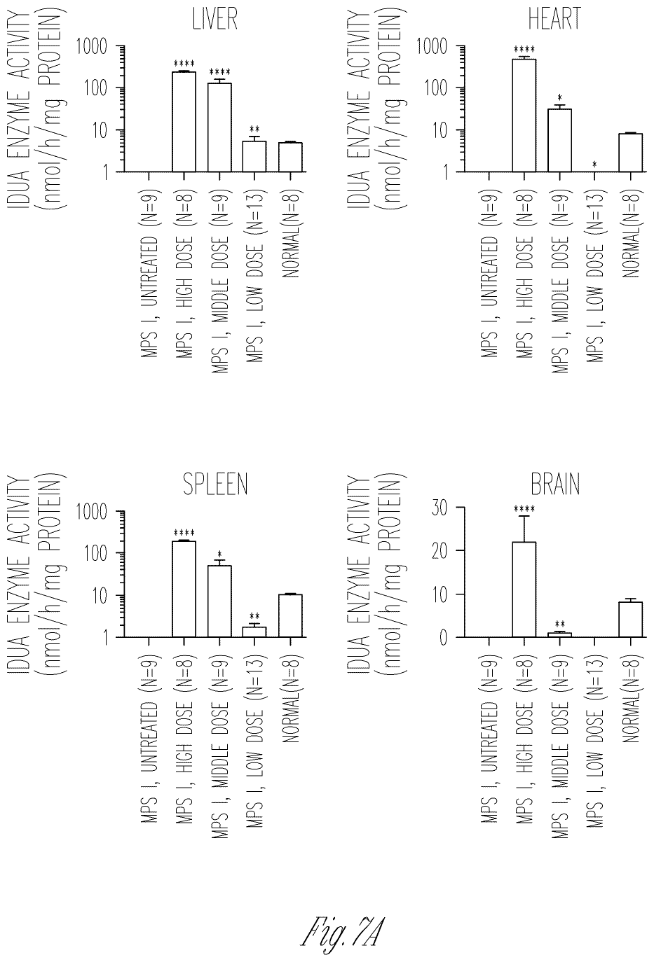

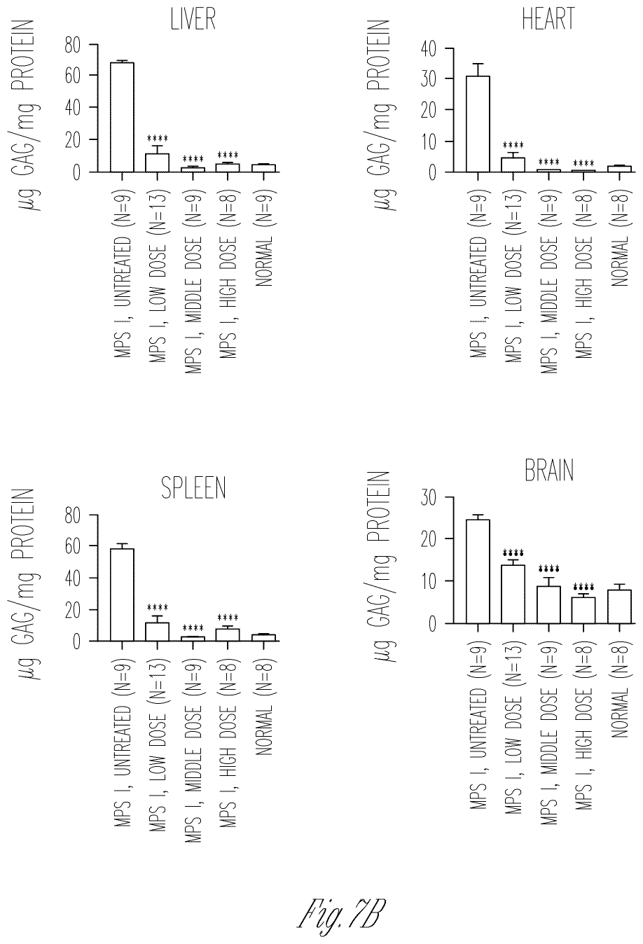

A total of 3 sgRNAs were designed to target the intron 1 of the human albumin gene. Then, the plasmids encoding these sgRNAs together with Cas9 were transfected into human hepatocytes (Huh-7 cell line). Sequencing results showed that Cas9 can be recruited to albumin intron 1 by sgRNA3 and then cut the DNA (FIG. 6A),

As part of the in vitro toxicology study, off-target analysis with GUIDE-seq, an unbiased method, is performed (Tsai et al., 2015). The traditional off-target analysis involves 2 steps: 1) predict potential off-target sites through in silico tools; 2) sequence the predicted sites. The in silico tools predict possible off-target sites across the genome based on the sequences of the genome and gRNA. Although off-target prediction algorithms have been improved over time, their genome-wide search criteria are not exhaustive. Therefore, this method is intrinsically biased. In contrast, GUIDE-seq relies on the integration of double-stranded oligodeoxynucleotides tag into DSB created by Cas9, and then search the whole genome for these tags (FIG. 6B). In this way, off-target sites can be identified in an unbiased manner. When tested in human hepatocytes (Huh-7 cells), no off-target cleavage of PS822 was identified through GUIDE-seq (FIG. 6C).

### The Minimal Effective Dose, Optimal Effective Dose, Time and Duration of Treatment

PS822 is not pharmacologically active at the target site in mice, and so studies in non-human cells use species-specific surrogate reagents, Therefore, surrogate PS822 reagents, which target the albumin locus in the mouse genome, were designed. In addition, since AAV2/8 vector has better liver tropism in mice. AAV2/8 vectors were used an pharmacology and toxicology studies performed.

TABLE 4
Group assignment for the pharmacology and
toxicology studies in mice.

| | Dose (AAV-IDUA and | |
|---|---|---|
| Group | AAV-Cas9 in vg/kg) | Group size |
| MPS I, high dose | 3 × 10¹⁴ and 5 × 10¹³ | 8 |
| MPS I, middle dose | 3 × 10¹³ and 5 × 10¹² | 9 |
| MPS I, low dose | 3 × 10¹² and 5 × 10¹¹ | 13 |
| MPS I, untreated | N/A | 9 |
| Normal heterozygotes, untreated | N/A | 11 |

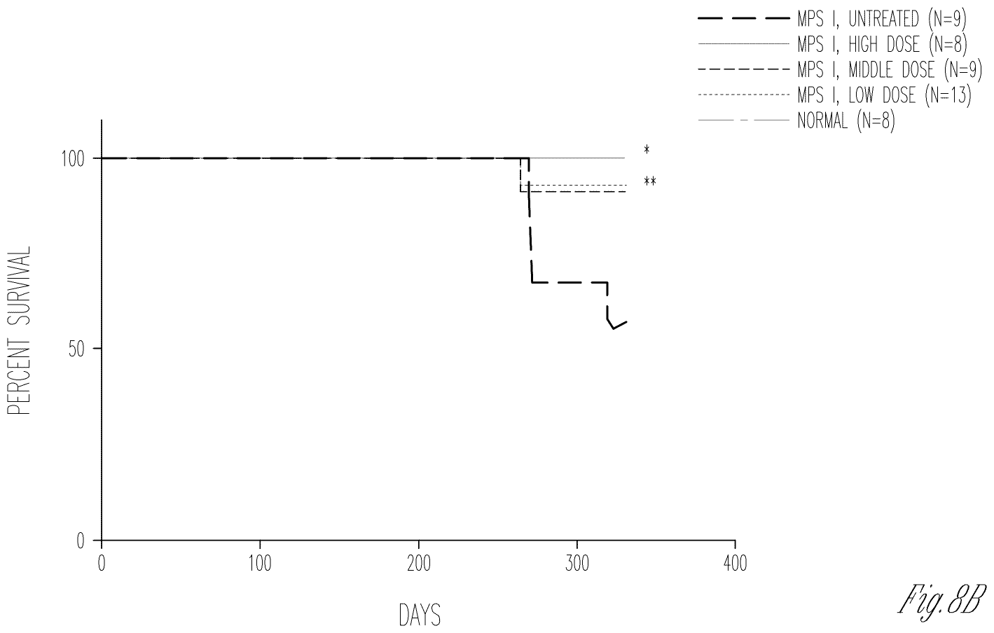

The PS gene editing system was assessed in MPSI mice for 11 months (lifespan of untreated MPS I and normal mice: 1 and 2.5 years, respectively). Neonatal MPS I mice received the surrogate PS822 reagents at 3 different doses. Group assignment is listed in Table 4. Plasma enzyme activity increased significantly in all treated mice (the high dose group achieved 700-fold of wildtype levels) and maintained for 10 months (next page, FIG. 8A). A comparison in plasma enzyme levels between this study and a previous ZFN study was performed. The middle dose of PS822 achieved 25-fold of peak levels in the ZFN study at <50% of the ZFN dose (3.5×10¹³ vs 7.5×10¹³ vg/kg). In general, treating neonatal mice may be less challenging due to immune tolerization effect. However, the adult mice were injected weekly with an immunosuppressant (cyclophosphamide), which in fact ameliorates the difference between the two studies. These results indicated that the PS system achieved magnitude (up to 50-fold) higher transgene expression.

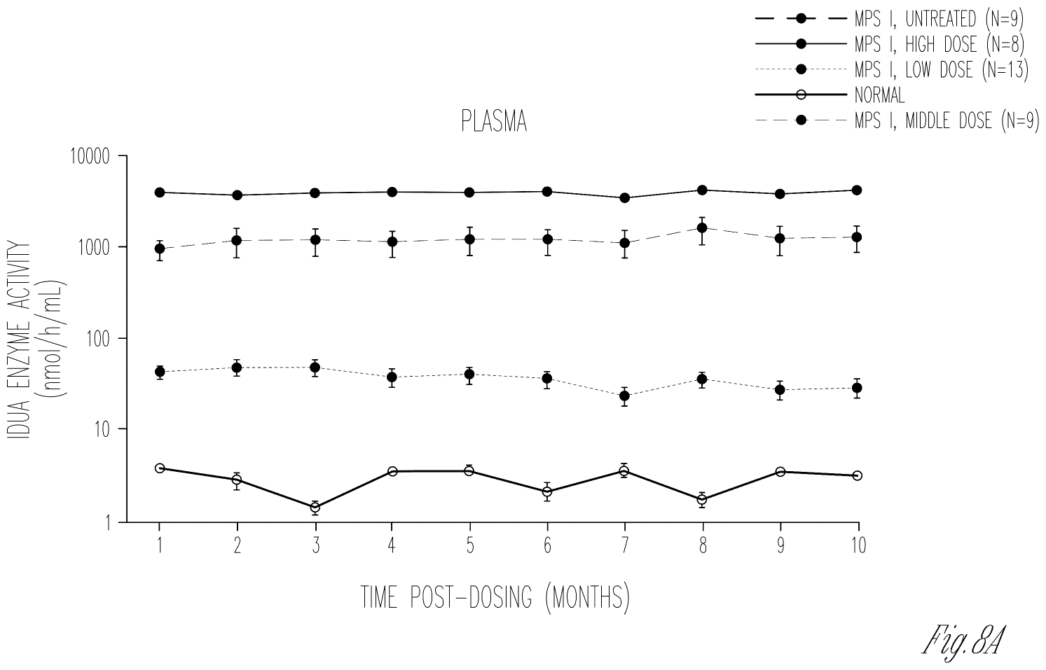

At 11 months post-dosing, tissues were collected from all mice after perfusion. There were significant enzyme activities (FIG. 7A) in tissues including the brain of high dose and middle dose treated mice in a dose-dependent manner. Further, there was significant GAG storage reduction (FIG. 7B) in the brain of all treated mice in a dose-dependent manner. Fear conditioning showed that treated mice had better memory and learning ability (next page, FIG. 8C). Pole test showed that treated mice had better coordination and motor function (FIG. 8D). Further, mice from all 3 treated groups had significantly better survival rates (FIG. 8B). These results showed that PS822 successfully treated systemic and neurological diseases of MPS I mice.

The dose-dependent relationship indicates that the higher the transgene expression, the more therapeutic benefits achieved. The minimal effective dose would be less than the low dose (total exemplary dose: 3.5×10¹² vg/kg) because the low dose group achieved significant GAG reduction in the brain, prolonged lifespan, and improved neurobehaviors. A more effective dose should be the middle dose (total exemplary dose: 3.5×10¹³ vg/kg) because the middle dose group achieved significant enzyme activity in the brain, normalization of GAG storage in the brain, prolonged lifespan, and improved neurobehaviors. Although the high dose group achieved higher enzyme activity than the middle dose group, it did not lead to further GAG reduction in the brain that was substantial. As shown in FIG. 7C, both middle dose and high dose group achieved normalization of GAG levels in the brain. Moreover, it is challenging to manufacture such amount of vector used in the high dose group. As to the time of treatment, as suggested by other groups, we believe that the earlier the treatment, the better the outcome is. A goal in the clinical trial is to treat human babies with MPS I to achieve maximal therapeutic benefits. The mice were followed for 11 months, and the normal lifespan of mice is around 2 years. Therefore, 11 months represent a very long-term follow-up relative to the lifespan of mice and the duration of treatment.

TABLE 5
Tumor rate in each group of mice.

| | Hepatocellular |
|---|---|
| Group | carcinoma |
| MPS I, high dose | 0/11 |
| MPS I, middle dose | 1/11 |
| MPS I, low dose | 0/13 |
| Normal mice (Goldsworthy | 6-13% |
| 2002; Donsante 2006) | |

As to the toxicology profile, PS822 was well tolerated in mice, with no test article-related unscheduled deaths, no clinical signs of toxicity, and no macroscopic or microscopic findings at necropsy. Histopathological analysis by identified hepatocellular carcinoma in only one mouse from the middle dose group (Table 5). Previous studies showed that normal mice at this age developed tumor at the rate of 6-13% (Goldsworthy & Fransson-Steen, 2002; Donsante et al., 2007). Therefore, there was no significantly increased tumor risk in treated mice. These results strongly support the long-term safety of PS gene editing.

### Mouse Model and Endpoints

In the in vivo pharmacology and safety murine studies, MPS I knockout mice (idua−/−) generated by Elizabeth Neufeld's group were used (Ohmi et al., 2003). The mouse model was generated by insertion of neomycin resistance gene into exon 6 of the 14-exon IDUA gene on the C57BL/6 background. The MPS I model series as a reliable model for patients with MPS I. A deficiency of the IDUA enzymatic activity in degrading GAG dermatan and heparan sulfate results in the accumulation of GAG in lysosomes of tissues including the brain, resulting in clinical manifestations of MPS I disease. GAG can also be detected in urine and tissues of MPS I mice, and thus can serve as biomarkers for disease progression. This mouse model has been used in our previous IND-enabling ZFN study and many other preclinical studies.

In nonclinical studies, the primary endpoint was GAG reduction in tissues, while the secondary endpoint was enzyme activity in tissues. Reduction of GAG in tissues and urine are the most frequently employed in IND-enabling studies to treat several MPS diseases. GAG reduction in tissues and urine were pharmacodynamic parameters in the INDs for ERTs approved for MPS II Hunter syndrome, MPS IVA, MPS VI, and MPS VII. Reduction of GAG in tissues and urine were also common efficacy outcomes in the clinical trials for these ERT. Therefore, the advantages of the GAG reduction in tissues is its frequent use in INDs and high clinical relevance in diseases similar to MPS I. It is becoming more widely recognized that the pathology of lysosomal diseases, including MPS I, is not limited to substrate accumulation. Therefore, the secondary endpoint of enzyme activity in tissues was used to capture other benefits from therapy.

Power analysis was performed based on pilot study data when deciding the sample size. A high effect size of 0.6 was estimated based on the relatively high editing efficiency seen in the ZFN study relative to the number of hepatocytes needed to edit to produce enzyme levels comparable to ERT (>0.5% of hepatocytes were edited with ZFN vs 0.05% of hepatocytes are needed to edit). Based on this effect size of 0.6, five groups, power of 0.8, and a significance level of 0.05, we calculated that 7.64 mice would be needed in each group, which was rounded up to 8 mice in each group.

Statistics were performed using Graphpad Prism 8 software. One-way ANOVA was performed to compare IDUA or GAG levels in different tissues between the heterozygotes, untreated MPS I, low dose treated MPS I, medium dose treated MPS I, and high dose MPS I. For survival analysis, log-rank test was chosen since we expect the relative risk to remain constant.

Randomization was done when assigning groups by flipping a coin. Behavioral tests were performed by a blinded veterinary technician. In a previous ZFN study, treated male mice had slightly higher IDUA enzyme activities in tissues than female mice, which has been observed in other AAV gene therapy studies (Nathwani et al., 2007). It has been shown that gender influences liver transduction efficiency of AAV vectors through an androgen-dependent pathway (Davidoff et al., 2003). However, in a preliminary mouse study with PS822, a substantial gender difference in therapeutic benefits (enzyme activities and GAG levels) was not observed.

### Bioavailability and the Presence of Enzyme in the Brain

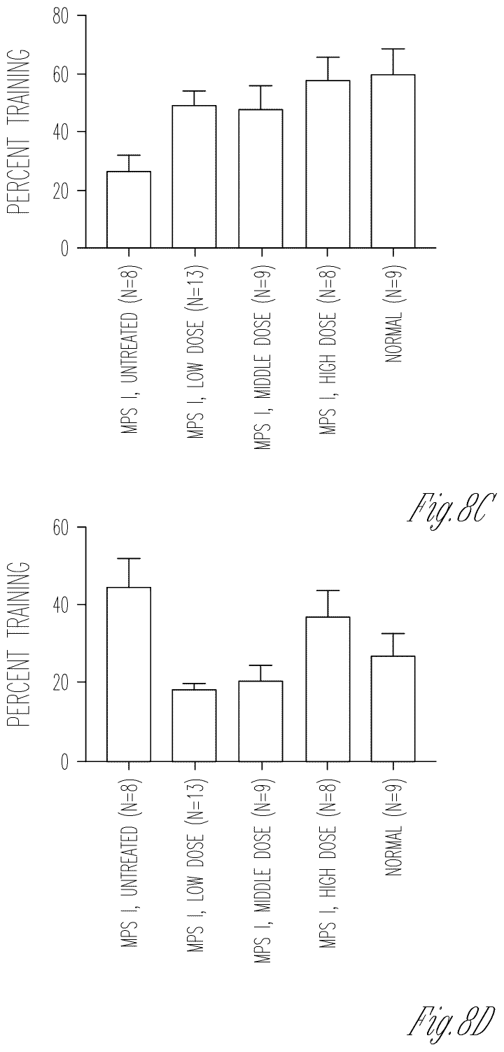

Given that the drug is administered intravenously, the PS system is not degraded in the intestines and therefore has an absolute bioavailability of 1. As shown in a previous ZFN study, although AAV vector was found in multiple tissues through QPCR, no gene editing events were observed outside the liver through deep sequencing. Therefore, only hepatocytes were edited to express the therapeutic transgene (Ou et al., 2019; Laoharawee et al., 2018). In this study, synthesized lysosomal enzymes secreted and reached multiple tissues, including the brain, through cross correction. The feasibility is supported by the gene editing studies and many high dose ERT studies from different groups (Table 2). Moreover, preliminary data with the PS gene editing system in MPS I and Sandhoff mice showed significant neurological benefits (FIGS. 7 & 8 for MPS I, FIG. 9 for Sandhoff). Like MPS I, Sandhoff disease is a lysosomal disease with neurological complications. These studies together showed that when there is a high and constant level of lysosomal enzyme in the blood, a small but sufficient amount can enter the brain.

### Data Interpretation

There is a possibility that AAV8 may have entered the brain and have edited the brain cells to express IDUA proteins. However, AAV8-Cas9 vector is under the control of a liver-specific TBG promoter. Even if some vectors enter the brain, editing of brain cells should not occur. Moreover, in a previous ZFN study, deep sequencing analysis of the brain samples showed no gene editing events outside of the liver (Ou et al., 2019; Laoharawee et al., 2018). Therefore, gene editing events outside of the liver are expected to be highly unlikely. Notably, the clinical trials with the ZFN that also target the albumin locus show no brain toxicity in all twelve patients (Muenzer et al., 2019; Harmatz et al., 2018). In the NHP pharmacology and safety studies, the gene editing events in tissues other than the liver are determined.

The PS system has a high effect size in relation to potential clinical impact. In the present study, a 50-fold higher efficiency was seen than using ZFN. This equates to 25% of albumin loci producing the therapeutic IDUA proteins. For a therapeutic effect equivalent to ERT, 0.05% of the albumin loci would need to produce the therapeutic protein. Therefore, the PS system achieves a 500-fold greater increase in the levels of therapeutic protein than the current treatment for MPS I. Since high levels of therapeutic protein have been shown to cross the BBB and reduce substrate storage in the brain, the PS system will be able to treat neurological complications of MPS I, which represents a great unmet medical need.

### Reproducibility of Data

In vivo pharmacology and toxicology studies of the PS gene editing system were conducted in another lysosomal disease, Sandhoff disease (SD). SD, a subtype of GM2-gangliosidoses, is a genetic disorder causing severe neurological diseases and premature death. SD results from the deficiency of a lysosomal) enzyme β-hexosaminidase A (Hex A) and subsequent accumulation of GM2 gangliosides.

The pharmacology and toxicology of the PS system was evaluated in neonatal SD mice (n=10). In this study, mouse surrogate PS813 reagents (AAV2/8-Cas9 at 5×10¹² vg/kg, and AAV2/8-HEXM-sgRNA at 3×10¹³ vg/kg) were used. Plasma Hex A enzyme activities in treated SD mice was markedly increased, up to 144-fold of wildtype levels (FIG. 9A). At 4 months post-dosing, tissues were collected from all mice after perfusion. Hex A enzyme activities in tissues, including the brain, were also significantly higher compared with untreated SD mice (FIG. 9B). Rotarod analysis showed that the coordination and motor function of treated mice were improved compared to untreated SD mice (FIG. 9C).

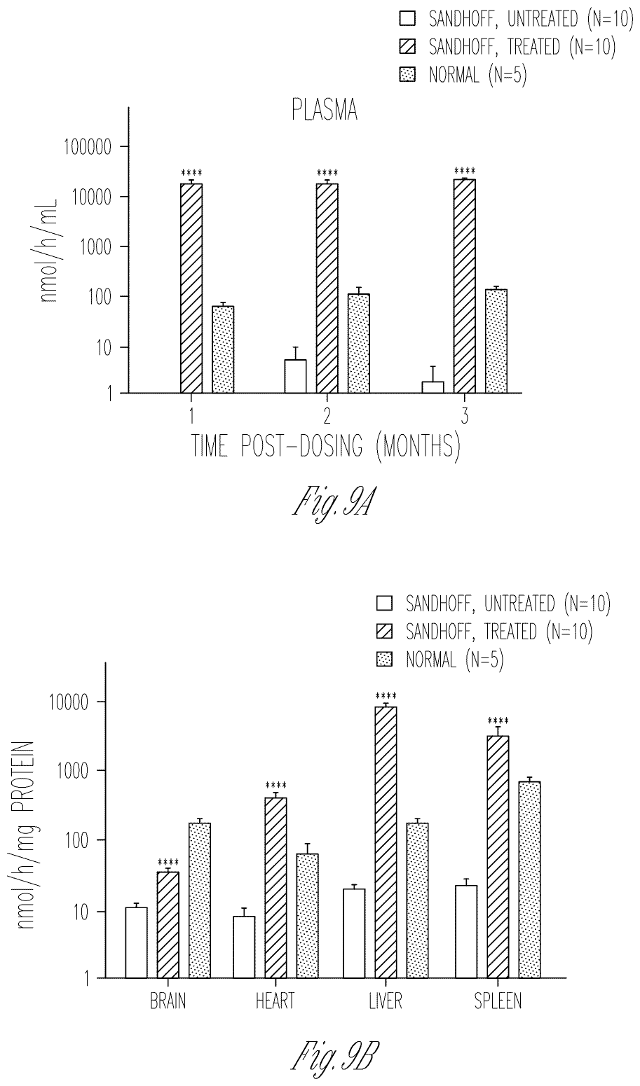

Cellular vacuolation is a typical microscopic observation of lysosomal diseases (Ohmi et al., 2013). Histological analysis of H&E stained tissues showed reduced cellular vacuolation in the brain and liver (FIG. 10, upper and middle panel) of treated SD mice. To test whether the enzyme expressed from the liver can enter the CNS, immunolabelling against Hex A was performed. There was an increased intensity of labeling that could be consistent with Hex enzyme in the brain of treated mice (FIG. 10, lower panel). These results further corroborate the findings of increased enzyme activities in the brain of treated mice. These results showed that the PS system achieved highly efficient transgene expression and substantial neurological benefits in SD mice. In terms of the safety profile, no morbidity events, mortality events or incidence of toxicity were observed in treated mice. In summary, these results showed that the PS gene editing system can efficiently treat neurological complications in multiple animal models of lysosomal diseases and also supported the reproducibility of the therapeutic benefits provided by the PS system.

The dose used is set as 5×10¹² vg/kg AAV2/6-Cas9 and 3×10¹³ vg/kg AAV2/6-hIDUA-sgRNA. All treated patients are evaluated for 36 months at 6-monthly intervals. The endpoints are summarized in Table 6. Inclusion/exclusion criteria, visit schedule and study procedures will also be specified. In addition, the safety monitoring and mitigation plan are determined, and the key potential anticipated risks include transaminitis due to cell-mediated immunity to capsid and/or AAV gene product (IDUA or Cas9), and reduction in albumin synthesis.

TABLE 6
Endpoints of the Phase I/II clinical trial.

Primary Endpoints:
Incidence of Treatment-Emergent Adverse Events (TEAEs) and Serious Adverse Events (SAEs)
[Time Frame: Up to 36 months after the infusion of the test article.]
Incidence of Treatment-Emergent Adverse Events (TEAEs) and Serious Adverse Events (SAEs) in
subjects who receive PS822 as assessed by Common Terminology Criteria for Adverse Events
(CTCAE)
Secondary Endpoints:
Effect of PS822 on IDUA activity [Time Frame: Up to 38 months after the PS822 infusion]
Change from baseline clinical laboratory in measurement of IDUA activity measured in blood (i.e.,
plasma, leukocytes), cerebrospinal fluid (CSF), and liver (percutaneous needle liver biopsy).
Effect of PS822 on urine GAG levels [Time Frame: Up to 36 months after the PS822 infusion]
Change from baseline in total GAG, DS GAG, and HS GAG (/creatinine ratio) measured in tissues
including blood, liver and CSF)
AAV2/6 clearance in plasma, saliva, urine, stool, and semen [Time Frame: Up in 36 months after the
PS822 infusion]
AAV2/6 clearance by measuring vector genomes in plasma, saliva, urine, stool, and semen by PCR.
Changes (or lack thereof) in:
Performance scores on the Bayley Scales of Infant Development
Opening pressure during lumbar spinal fluid pressure.
Height, weight and occipofrontal head circumference (OFC).
Brain volumes (total, white matter, grey matter) as measured by MRI volumetrics.

### In Vitro Cas9 Cutting Kinetics and Activity

Methods: In vitro pharmacokinetics of Cas9 cutting efficiency is evaluated in human hepatocytes. Gene modification levels in hepatocytes after the treatment with PS822 at different doses is evaluated over 10 days of exposure. Cells are harvested on Days 1, 3, 5, and 10, genomic DNA will be isolated, PCR amplified and MiSeq deep sequenced. The primary endpoint will be % indels because it can reflect the cleavage activity of Cas9 at the target locus.

A dose- and time-dependent increase in cutting efficiency is observed (measured as % indels).

### Characterization of the Integrated Sequence at the Target Locus in MPS I Mice

Rationale: As shown in FIG. 5, gene editing events at the target include HDR-mediated insertion, NHEJ-mediated insertion, and NHEJ-mediated indels. Since MiSeq can only measure the frequency and extent of indels, it represents an indirect measurement of gene editing events at the target locus. Therefore, it will be important to characterize the on-target gene modification events.

Methods: The genomic DNA has been extracted from liver samples of mice treated with high dose, middle dose, and low dose of PS822. MiSeq will be performed to determine % indels at the target locus. The enzyme activities obtained in the preliminary study will be used to determine the correlation between % indels and enzyme activities. Further, the on-target gene editing events are characterized through a ligation-mediated PCR (LMU-PCR) method coupled with unique molecular indices followed by deep sequencing.

A positive correlation is observed between % indels and hIDUA levels, which will provide information for dose selection in the clinical trial. Further, LMU-PCR and deep sequencing analysis will determine the HDR-mediated gene targeting efficiency. Similar to observations from previous studies, insertion of AAV genome sequence at the target locus is expected. In addition to the HDR-mediated transgene insertion, ITR and other elements of AAV genome sequence are observed indicative of NHEJ-mediated insertion.

### Immunogenicity Studies in Adult MPS I Mice

Rationale: The immune system of human subjects in the PS822 clinical trial may be exposed to several antigens arising from the administration of PS822. Immunogenicity of human IDUA proteins in toxicology species (mouse and cynomolgus monkey) may not be predictive of an immune response in human subjects, therefore, the assays are considered to be of minimal value. A previous study showed that there could be immune responses against Cas9 proteins (Nelson et al., 2019), which could affect the transgene expression. In addition, although AAV is a replication-defective virus, humans could be naturally infected during childhood. Therefore, pre-existing neutralizing antibodies to AAV may affect transduction by forming immune complexes with the vector. Further, memory CD8 T cells may be reactivated and eliminate transduced hepatocytes that express AAV protein-derived epitopes or Cas9 proteins. Results from AAV clinical studies suggest that a period of immunosuppression (e.g., corticosteroids) during the period when AAV-derived epitopes are being presented may be necessary to achieve sustained IDUA expression. Therefore, immunogenicity assays for AAV vectors and Cas9 proteins in MPS I mice will be performed.

Methods: MPS I mice (n=12) receive IV administration of mouse surrogate PS822 reagents at the dose of 3.5×10¹³ vg/kg (middle dose) at 1 to 2 months of age. Half the mice receive corticosteroids, and the other half of mice do not receive corticosteroids to see if immune responses can be modulated. Plasma samples are collected from treated mice and controls biweekly for ELISA. ELISA for antibody against Cas9 proteins are performed, and ELISA for antibody against AAV2/8 vectors. At necropsy, splenocytes from injected mice are isolated and purified for ELIspot to evaluate T-cell responses to AAV and Cas9. Moreover, plasma and tissue enzyme activities are measured to see if the efficacy of gene editing has been affected.

T-cell responses and antibodies to AAV2/8 or Cas9 develop in mice that do not receive corticosteroids. T-cell responses and antibodies to AAV2/8 or Cas9 do not develop in mice that receive corticosteroids. In the unlikely event that corticosteroids do not attenuate immune responses, bortezomib is used.

### Biodistribution from a 90-Day Study in Cynomolgus Monkeys

Rationale: The primary objectives of this study in immunosuppressed normal cynomolgus monkeys will be to assess the pharmacokinetics and biodistribution of PS822. Due to the sequence difference at the target locus between monkey and human genome, species-specific surrogate PS822 reagents will be used in the NHP studies. The NHP surrogate reagents are the same as human PS822 except that the sgRNA and homology arm sequences are changed to target the cynomolgus monkey albumin intron 1 locus. The NHP studies include multiple components including pharmacokinetics, biodistribution, pharmacology, and toxicology, but only pharmacokinetics and biodistribution experiments are discussed herein.

AAV2/6 vectors have similar distribution properties, dependent on the AAV6 capsid proteins (Zincarelli et al., 2018; Wang et al., 2010; MacLachlan et al., 2013). AAV6 has reproducible liver tropism, demonstrated in rabbits, mice, dogs, and NHPs (Zincarelli et al., 2018; Wang et al., 2010; MacLachlan et al., 2013; Favaro et al., 2011; Nathwani et al., 2006; Nathwani et al., 2002; Jiang et al., 2006; Stone et al., 2008). Therefore, we expect that the biodistribution of AAV2/6 vectors will be similar to previous studies. Nevertheless, biodistribution data is collected from cynomolgus monkeys, and we expect the biodistribution of AAV2/6 vector in monkeys to be highly relevant to clinical trials.

Methods: GMP-comparable AAV2/6 vectors are provided by CHOP Clinical Viral Vector Core. The NHP experiments are performed by Envol Biomedical, which has been providing high-quality services including NHP pharmacokinetics, toxicology, and pharmacodynamics studies. Animals are prescreened for neutralizing antibodies against Cas9 and AAV6 capsids. Enrolled animals are randomized into two groups at the injection day by flipping a coin, and blinding is performed (the group assignment will be unknown to the CRO staff). Two groups of male cynomolgus monkeys receive a single IV infusion of the test article or formulation buffer into a peripheral vein using a calibrated infusion pump (target rate=1 mL/min) on Day 1. Dose and group assignment information is listed in Table 7. To mitigate a potential immune response against the AAV capsid and/or hIDUA proteins, all animals receive the immunosuppression regimen

TABLE 7
Pharmacokinetic and biodistribution studies in NHP.

| Study | Animals | Test article, dose, route | Evaluations |
|---|---|---|---|
| A 90-day study to | Cynomolgus | Treatment group receive | Pharmacokinetics |
| evaluate pharmacology | monkey | AAV2/6-Cas9 | AAV pharmacokinetics (pre-dose, 30 |
| and toxicology following | Treatment group: | (5 × 10¹² vg/kg) | min, and 24, 48, 144 and 312 hours |
| IV administration of | 3 Male/group | AAV2/6-IDUA-spRNA | post-dose) through QPCR; |
| surrogate PS822 via | Control group: | 3 × 10¹³ vg/kg) | AAV biodistribution (brain, liver, lung, |
| AAV2/6 vectors in | 3 Male/group | and | spleen, heart, kidney, lymph node, |
| cynomolgus monkeys | | Immunosuppression regimen: | intestine, testes, and stomach) through |
| | | 10 mg/kg methylprednisolone, | QPCR; |
| | | IM on Days 5-90 and 10 | Shedding analysis (saliva, semen, urine, |
| | | mg/kg rituximab IV on Days 7 | feces) through QPCR; |
| | | and 14 | Cas9 biodistribution (brain, liver, lung, |
| | | Control group only receive | spleen, heart, kidney, lymph node, |
| | | formulation buffer and the | intestine, testes, and stomach) through |
| | | immunosuppression regimen | RT-QPCR. |

To determine the pharmacokinetics of AAV2/6 vectors, plasma samples will be collected at different timepoints (pre-dose, 30 min, and 24, 48, 144, and 312 hours post-dose).

To assess the biodistribution, periodic liver biopsies (Days 14, 37, 63) and necropsy at Day 90 are performed for quantification of AAV vector copy numbers in liver samples using qPCR. In addition, biodistribution in other tissues including adrenal gland, brain (cerebellum and frontal cortex), heart, kidney, liver, lung, spleen, and testes samples will also be determined, The lower limit of quantification (LLOQ) for the QPCR assay is determined prior to studies.

AAV2/6 shedding analysis is conducted with cynomolgus monkey biological fluids (saliva, urine, and feces) for AAV-Cas9 and AAV-IDUA on Days 1 (predose), 2, 4, 12 and Days 58-61.

Cas9 biodistribution in tissues (brain, liver, lung, spleen, heart, kidney, lymph node, intestine, testes, and stomach) is determined by measuring Cas9 mRNA levels via RT-QPCR.

The results show that the plasma concentrations of both Cas9 and hIDUA vector in treated animals peak at an interim timepoint and decline abruptly. The values for Cmax, AUC(0-infinity), and half-life are determined. No copy number is detected in control animals. These results are used to quantify clearance (CL) and volume of distribution (Vd) and show the pharmacokinetic profile of PS822 in NHP.

As to copy number in the liver, vector genome copies are detected in treated animals at each time point, with highest levels generally found on an interim timepoint. By Day 90, copy numbers of both the Cas9 and hIDUA vector are decreased. No copy number is detected in the liver of control animals.

As to biodistribution, in control animals, no Cas9 nor hIDUA vector are detected in DNA isolated from adrenal gland, brain (cerebellum and frontal cortex), heart, kidney, liver, lung, spleen, and testes samples. In treated monkeys, high levels of hIDUA and Cas9 DNA will be detected in the liver. The levels of hIDUA and Cas9 vector are determined in adrenal gland, brain (cerebellum and frontal cortex), heart, kidney, liver, lung, spleen, and testes samples.

No Cas9 nor hIDUA vectors is detected in feces, saliva, and urine samples from treated animals collected on Day 1 (predose). At post-dosing timepoints, hIDUA and Cas9 vector are detected in feces, saliva, and urine samples from treated animals and gradually reduce over time. No vectors are detected in control animals.

Cas9 mRNA is found only in the liver of treated animals. These results indicate that the Cas9 catalytic activity is limited to the liver through the liver-specific TBG promoter and the liver tropism of AAV2/6 vectors. In addition, the Cas9 mRNA level decreases gradually over the time, similar to previous studies (Nelson et al., 2019; Yang et al., 2016).

### Additional In Vitro Toxicology Studies

### Determine the Transformation Potential of PS822 In Vitro

Rationale: This study is to evaluate the transformation potential of PS822 by evaluating the anchorage-independent growth of a genome modified human WI-38 cell line in soft agar media following genome modification by PS822. WI-38 is an adherent human diploid lung fibroblast cell line that shows anchorage-dependent growth. Tumor growth and invasion is a complex process that involves anchorage-independent growth, motility, and degradation of the extracellular matrix. These processes can be simulated in vitro by measuring the ability of a cell to grow independently of substrate adhesion and form colonies in a soft agar matrix (Shin et al., 1975). Since the growth of normal cells is anchorage-dependent while transformed cells lose this constraint and grow in an anchorage-independent manner, transformed cells can be easily differentiated from normal cells.

Methods: The study design is similar to the IND-enabling experiments for the ZFN study. WI-38 ceils are transduced with AAV2/6 vectors, and transduced cells from each condition are analyzed for % indels at the target locus by MiSeq at 5 days post-dosing. Integration of the hIDUA donor at the target locus will be confirmed by PCR. Modified cells are plated at two concentrations (1⁵ and 1⁶ total cells/plate) together with positive (transformed human cell line HT-1080 fibrosarcoma cells) and negative controls (non-transduced WI-38 cells).

No anchorage-independent growth are observed in treated cells and negative controls, while positive controls (HT-1080 cells) exhibit growth. In conclusion, gene modification of WI-38 fibroblasts by PS822 does not promote tumorigenicity in vitro.

### Off-Target Analysis Through in Silica Prediction and Targeted Sequencing

Rationale: As previously described, unbiased off-target analysis was performed through GUIDE-seq and no significant off-target effects of the PS822 in human hepatocytes was found. Since the detection limit of GUIDE-seq is 0.1% (Tsai et al., 2015), targeted sequencing at the predicted sites is a good complement due to its superior detection limit of 0.01% (Hendel et al., 2015). Therefore, in silico prediction is performed and then MiSeq sequencing of predicted sites conducted in human hepatocytes.

Methods: Top off-target sites are predicted by the Benchling software (Uniyal et al., 2019). To determine if these sites are cleaved by PS822, the human hepatocytes are transduced with different doses of PS822. Then, targeted sequencing at the predicted sites by MiSeq is performed.

No significant indels % are detected at any of the potential off-target sites. In the unlikely event of high off-target activity identified at a site with important biological function, the gRNA is redesigned to target the albumin locus and then tested in human hepatocytes.

### Additional In Vivo Pharmacology and Toxicology Studies in NHP with GMP-Comparable Vector

Rationale: The pharmacology and toxicology studies in NHP use GMP-comparable AAV2/6 vectors to deliver PS822 surrogate reagents. These studies evaluate gene modification at the albumin locus and hIDUA expression and include standard toxicology assessments.

Methods: The GMP-comparable vector is produced by CHOP Clinical Vector Core. The study design is listed in Table 8.

To suppress the potential immune response, an immunosuppression regimen is employed. The toxicology experiments include assessment of clinical signs, food consumption, body weights, clinical pathology (hematology, clinical chemistry, coagulation, and urinalysis), full necropsy (including macroscopic examination, and recording organ weights), and histopathological analysis of tissues. If off-target sites are found in the in vitro toxicology studies, targeted sequencing is performed at the homologous site in the liver of treated monkeys.

TABLE 8
Pharmacology and toxicology studies in NHP.

| Study | Animals | Test article, dose, route | Evaluations |
|---|---|---|---|
| A 90-day study to | Cynomolgus | Treatment group receive | Pharmacology |
| evaluate | monkey | AAV2/6-Cas9 | Periodic liver biopsies and necropsy for on- |
| pharmacodynamic and | 1-3 Male/ | (5 × 1012 vg/kg) | target and off-target gene modification (Miseq); |
| biodistribution following | group | AAV2/6-IDUA-sgRNA | Plasma, liver and PBMC IDUA activity; |
| IV administration of | | (3 × 1013 vg/kg) | hIDUA transgene mRNA expression levels in |
| surrogate PS822 via | | and | liver by RT-PCR; |
| AAV2/6 vectors in | | Immunosuppression regimen: | Toxicology |
| cynomolgus monkeys | | 10 mg/kg methylprednisolone, | Mortality, clinical observations, food |
| | | IM on Days 5-90 and 10 mg/kg | consumption, bodyweight, gross necropsy |
| | | rituximab IV on Days 7 and 14 | observations, organ weights; |
| | | Control group only receive | Hematology, clinical chemistry and coagulation |
| | | formulation buffer and the | parameters; |
| | | immunosuppression regimen | Pariodic liver biopsies and necropsy |
| | | | Microscopic evaluation of tissues |

In addition, the pharmacology profile is determined. Gene modification events at the albumin locus, IDUA enzyme activity in liver, plasma and PBMCs, and characterization of the albumin-hIDUA fusion transcripts are endpoints of this study.
1. All animals will survive the study duration without test article related clinical signs or changes in food consumption or body weight.
2. No changes in coagulation parameters.
3. There may be minimal to moderate immunosuppressant-related changes: increase in alanine aminotransferase (ALT) and aspartate aminotransferase (AST); increase in platelets; decrease in eosinophils and lymphocytes; biliary hyperplasia; multifocal periportal mononuclear cell infiltrates; lymphoid depletion; hepatocellular refraction and increased hepatocyte multinucleation in liver biopsies; reduced thymus and spleen weights, grossly small spleens.
4. No other definitive test article related histopathological findings in any tissue examined.
5. Robust on-target gene modification rate.
6. Increased IDUA enzyme activity in liver, plasma, and PBMCs.
7. Albumin-hIDUA fusion transcript will be identified through RT-QPCR.

The test article is well tolerated in cynomolgus monkeys without test article related adverse events or toxicity. The monkey surrogate PS822 reagents efficiently edit the target locus, and successfully express therapeutic proteins. These results in cynomolgus monkeys provide valuable information about the pharmacology and toxicology of PS822 in large animals (NHP).

Exemplary Embodiments

In one embodiment, a method to prevent, inhibit or treat a disease in a human is provided. The method includes administering to the human an effective amount of i) Cas or an isolated nucleic encoding Cas, and ii) isolated nucleic acid for one or more gRNAs comprising a targeting sequence for a human genomic target and nucleic acid comprising a coding sequence for a prophylactic or therapeutic gene product flanked by homology arms that bind to the human genomic target, or an effective amount of iii) isolated nucleic encoding Cas and nucleic acid for one or more gRNAs comprising a targeting sequence for a human genomic target, and iv) isolated nucleic acid comprising a coding sequence for a prophylactic or therapeutic gene product flanked by homology arms that bind to the human genomic target. The expression of the coding sequence in the human prevents, inhibits or treats the disease. In one embodiment, the disease is mucopolysaccharidosis, a lysosomal storage disease, hemophilia, thalassemia, or sickle cell disease. In one embodiment, the targeting sequence or homology arms are targeted to an intron. In one embodiment, the intron is an albumin gene intron. In one embodiment, the intron is the first intron. In one embodiment, one or more adeno-associated virus (AAV), adenovirus or lentivirus is/are employed to deliver at least one of the molecules of i) or ii) or at least one of molecules of iii) or iv). In one embodiment, a first rAAV delivers nucleic acid encoding SpCas9. In one embodiment, a second rAAV delivers the nucleic acid comprising the targeting sequence and the coding sequence. In one embodiment, the first or second AAV is one of serotypes AAV1-9 or AAVrh10. In one embodiment, the first and the second rAAVs are different serotypes. In one embodiment, one or more of the gRNAs target an albumin locus, Rosa26 locus, BCR locus, AAVS1 locus, CCR5 locus, HPRT locus, or alpha fetoprotein locus. In one embodiment, the disease is mucopolysaccharidosis type I, type II type III, type IV, type V, type VI or type VII. In one embodiment, the coding sequence encodes iduronidase, beta-globin, iduronate, beta galactosidase, sulfatase, arylsulfatase B, hexM, hexoaminidase A or hexosaminidase B. In one embodiment, the targeting sequence targets sequences within the first 500, 400, 300, 200, or 100 nucleotides of the intron. In one embodiment, the Cas comprises Streptococcus pyogenes (SpCas9), Staphylococcus aureus (SaCas9), Streptococcus thermophilus (StCas9), Neisseria meningitidis (NmCas9), Francisella novicida (FnCas9), Campylobacter jejuni (CjCas9), CasX and CasY, Cas12a (Cpf1), Cas14a, eSpCas9, SpCas9-HF1, HypaCas9, Fokl-Fused dCas9, or xCas9. In one embodiment, liposomes are employed to deliver i), ii), iii), iv), or any combination thereof. In one embodiment, the nucleic acid comprising a coding sequence, e.g., for a prophylactic or therapeutic gene product, is not operably linked to a promoter in the nucleic acid to be delivered. In one embodiment, the gRNA is targeted to a region that is not polymorphic. In one embodiment, the gRNA is targeted to a region that is polymorphic, e.g., a region having a genomic nucleotide sequence that is only present in a subset of humans. In one embodiment, at least one homology arm is targeted to a region that is not polymorphic. In one embodiment, at least one homology arm is targeted to a region that is polymorphic, e.g., a region having a genomic nucleotide sequence that is only present in a subset of humans. In one embodiment, the polymorphism comprises 1291543917, rs555168961, rs1005433164, rs573310978, rs1201092701, rs1309281661, rs124952753, rs916755134, rs1297986401, rs540536260, rs1044205877, rs1321823482, rs1424193509, rs1015196134, rs1439794145, rs1160490434, rs1160928232, rs1441491010, rs1378384299, rs969133603, rs1176450394, rs898812665, rs750272107, rs973125757, rs1218941389, or rs930334301. In one embodiment, at least one homology arm is mutated relative to the genomic sequence in the human genomic target. In one embodiment, at least one homology arm has 100% sequence identity to the genomic sequence in the human genomic target. In one embodiment, rAAVs deliver the components and the gRNA and the homology arms are specific for the first intron of the human albumin gene, wherein the targeting sequence targets sequences within the first 500, 400, 300, 200, or 100 nucleotides of the intron and the Cas is not SaCas9.

In one embodiment, a composition is provided comprising a first vector comprising an isolated nucleic encoding Cas9 and a second vector comprising an isolated nucleic comprising sequences for one or more gRNAs comprising a selected human genomic targeting sequence and a selected coding sequence flanked by homology arms that bind to the human genomic target, or a first vector comprising an isolated nucleic encoding Cas9 and an isolated nucleic comprising sequences for one or more gRNAs comprising a selected human targeting sequence and a second vector comprising a selected coding sequence flanked by homology arms that bind to the human genomic target, wherein at least one of the homolog arms is mutated. In one embodiment, the vector is a rAAV vector. In one embodiment, the targeting sequence targets intron 1 of the human albumin locus. In one embodiment, the Cas is SpCas.

In one embodiment, a method to detect neurological inflammation or neurological impairment in a Mammal is provided. The method includes detecting chitotriosidase activity in a cerebrospinal fluid sample of a mammal with a lysosomal storage disease, wherein increased chitotriosidase activity in the sample relative to a corresponding sample from a control mammal is indicative of neuroinflammation or neurological impairment. In one embodiment, the mammal is a human, in one embodiment, the disease is gangliosidosis. In one embodiment, the disease is mucopolysaccharidosis. In one embodiment, the mammal has been subjected to enzyme therapy or gene therapy for the lysosomal storage disease.

In one embodiment, a method to decrease Cas9 activity on a nucleic acid template having two homology arms specific for a locus in a mammalian genome is provided. The method includes introducing into a mammalian cell a nucleic acid template having two homology arms each flanking a nucleotide sequence of interest, wherein at least one of the homology arms is mutated, and wherein the cell comprises Cas9 and a gRNA. In one embodiment, the locus is a human locus. In one embodiment, the locus is the albumin locus. In one embodiment, at least one of the homology arms has at least 7 mutations. In one embodiment, the mutations are in a 20 to 30 contiguous base pair region of the homology arm. In one embodiment, the region is adjacent to the nucleotide sequence of interest.

The invention will be described by the following non-limiting examples.

### EXAMPLE 1

Since the human albumin intron locus is a target for gene editing, which is not conserved between mouse and human, a series of human gRNA sequences was tested.

An exemplary human albumin genomic sequence is shown below:

```
(SEQ ID NO: 27)
      1 tttattgttt tcaatatcta tataatgaaa aactaatact gaacaattca atgcttatat
    61 acccaaaaat attttacaat tatttttgaa agcatttata ttatgtagtt tattataatt
  121 tggctagaga tgaatttctc tcaaaaaagt gaacatctgc caattattta tatgatatat
  181 ttcttaaaac ttacactttt ttcgatcaga taattcattt attgatgtca gcgcacacat
  241 accttcttgg aaaactcctg agaatttcaa ggcttagatt ccaagcttca gctgggttac
  301 gggttgagaa agttttagct aagaactggc actaaaagat tacaaaacat ctcgcaaagc
  361 ttaaagctat agaagacatc aagaagaaaa aatacctttt aataaaaaca ttctaaactc
  421 tatccactaa aatttcaaca tgagactatt ttagctatac aatcttagtt gttttataga
  481 agattgataa agttcaattt tataacttta aacttaagta acaagtttct tgaatttcat
  541 gtttaaaata tagaatatga atagattcaa agtgcattca tttctgttta atttccatct
  601 gtggctatcc ttatgagaca ggattagtat taatttcaat ttatatgttt atagattcta
  661 ttagttgtta atttattaag aatggataaa catggattgc agacattaac tatatttttc
  721 ttgcacttga aaaacttgat aaaaaaatat gagaaagtct cattaatata gtttaaataa
  781 gtttaaatga actttttatt ctttaaaaat attaagctga actagctaga gcgtaatact
  841 tgaggagcaa ttgtgaggct taggaatgaa caagctctca aacttgaatc ctaattgcct
  901 aacttgatat attctttcct accactcagg tccttttctc atttgataag gctgtcacat
  961 tgaaaacatg gaattgcaga atgttcaagg tagaaggatc gttaaagata atttagtcca
1021 acttgaccca tatggcatag agatatatgc agatctgcat gtccagttct gcagctagaa
1081 actgacactg agctgaatta aaccacacgg agaagaactg agagatttaa agtaaaaggt
1141 taggaaattc gaatgtatat atattccaga ggcccagtgt aacatgaaga aagtcaagga
1201 gagggactct ttcatcatgg aagatctggg ggcagaggga cttaaaaggt gggacaagcc
1261 aaagatggtg acctagggtg gtttttatga tataaccttt tttctcctca ttccctatga
1321 aatcttcagc caattccata tttctcacta attcaagtga atatctgggg ggatggctta
1381 tggagggaaa taagggagca attgtttgga ttatattcca attttttatt tgcattagaa
1441 tacctatatg tttatacatt tttatttgcg ttagaaatac ttatataagt ttatacactt
1501 tcattctaac tttttttgag ggaaatgtat ctccataagt gaataagaag ttaaattact
1561 gattatgttt caattggtta gtttgatgct taacagtctt tcacttttcc actcttgcat
1621 attttgtaga gagtaataca agctcttcat gataggaagg atctgcatat gtttatttta
1681 tagtagaaaa atggtaagat tttcctgtcc ccaattttct ctccaaacaa atccaaattt
1741 cttgtatttt ggaatgactc tgggtaaagt ggttgtttat agaaagtctc ttataaaata
1801 ttgaggatat tattagatat tattacattt ccatgaatat gtttcccttt atctttaaga
1861 gaagaagagt cagaaatata acctatcaga gtgggagatg gcataaaagc tggactaaat
1921 ggattgctag atggagggca aacctggtgt gaatgactca gtgagaagct tcgggagtcc
1981 tgagggtagc agaagggtgc ggatttaaag ttactgttag agtggctgga aaatgggaga
2041 ccggttcaga gacattttat ctacttaaaa actgtgcctt ttgtatcacg tcaaagtgaa
2101 tgcaaaacaa agaacagaag ggttaaaggc tcaggtttaa atcccaggta tatgtacatt
2161 tcaattgagg tatttttttt ttcttttcta aatgatcagt acacttattc tttctaaaga
2221 aaatactttt cttaactact ctctattttt aaacttctcc cacaaagatg agaaaacatt
2281 taaaaatcat tggggctatt tttctgttta ccgagtaaag agaatctcta aaccatattt
2341 ataactctta ctctaaatat ttgcatttac cctcatgcca gagcccgttg atgactgact
2401 aaacagagtt tcaaagtttg aagaacagga aatttagaaa tgactaacaa ttatgtaggt
2461 ttatttctct cagtatagaa tgttcatata gaattaatgc cagaggtttt cagagaaaaa
2521 tgcagaaatt tttactttgc aaatccagaa gatgcaattg ttcaagtatt tcttaagaaa
2581 cattaatttt aagtatgcag atatcattga gaattaaata ttttaatttc taaactatta
2641 atcttttagt aggatgcaca tatgcaaaat gcctcattag tactgtaaga aaagattctt
2701 ggccgggcgc ggtggctcat gactgtaatc ccagcacttt gggaggccga ggtgggcgga
2761 tgacgaggtc aggagatcga gaccaccctg gcacacggtg aaaccccgtc tctactagag
2821 atacaaaaaa ttagccgggc gtgatggcgg gcgcctgtag tcccagctac tcgggaggct
2881 gaggcagaag aatggcgtga actcgggagg cggagcttgc aagtgagccg agatagtgcc
2941 actgcactcc agtctgggcg aaagagcgag actccatctc aaaaaaaaaa aaaaaaaaag
3001 aaaagattct tttaggtttc atcaattttg ttttaaagct agggctcttc attagatata
3061 ggaaaatcaa ttcaaagttt ctattcagtc atgatgaatt tgagattttt ttaggtttct
3121 Ctgtatttaa caatatatta cattataatg ttgtggtgaa aactaaatgg actaatatta
3181 ttcttttcat ttgttaaatg aaaaagtatg cacaaagtat atgtgagagt gacaaaggcc
3241 tgaatttgtc aattagtaac aattgtattc aacagtaagg attttatgtt tgggtaggcc
3301 tttcccaggg acttctacaa ggaaaaagct agagttggtt actgacttct aataaataat
3361 gcctacaatt tctaggaagt taggagttga cataatttat ccaagaaaga attattttct
3421 taacttagaa tagtttcttt tttcttttca gatgtaggtt tttctggctt tagaaaaaat
3481 gcttgttttt cttcaatgga aaataggcac acttgtttta tgtctgttca tctgtagtca
3541 gaaagacaag tctggtattt cctttcagga ctcccttgag tcattaaaaa aaatcttcct
3601 atctatctat gtatctatca tccatctagc tttgattttt tcctcttctg tgctttatta
3661 gttaattagt acccatttct gaagaagaaa taacataaga ttatagaaaa taatttcttt
3721 cattgtaaga ctgaatagaa aaaattttct ttcattataa gactgagtag aaaaaataat
3781 actttgttag tctctgtgcc tctatgtgcc atgaggaaat ttgactactg gttttgactg
3841 actgagttat ttaattaagt aaaataactg gcttagtact aattattgtt ctgtagtatc
3901 agagaaagtt gttcttccta ctggttgagc tcagtagttc ttcatattct gagcaaaagg
3961 gcagaggtag gatagctttt ctgaggtaga gataagaacc ttgggtaggg aaggaagatt
4021 tatgaaatat ttaaaaaatt attcttcctt cgctttgttt ttagacataa tgttaaattt
4081 attttgaaat ttaaagcaac ataaaagaac atgtgatttt tctacttatt gaaagagaga
4141 aaggaaaaaa atatgaaaca gggatggaaa gaatcctatg cctggtgaag gtcaagggtt
4201 ctcataacct acagagaatt tggggtcagc ctgtcctatt gtatattatg gcaaagataa
4261 tcatcatctc atttgggtcc attttcctct ccatctctgc ttaactgaag atcccatgag
4321 atatactcac actgaatcta aatagcctat ctcagggctt gaatcacatg tgggccacag
4381 caggaatggg aacatggaat ttctaagtcc tatcttactt gttattgttg ctatgtcttt
4441 ttcttagttt gcatctgagg caacatcagc tttttcagac agaatggctt tggaatagta
4501 aaaaagacac agaagcccta aagtatgtat gtatgtatat gtgtgtgtgc atgcgtgagt
4561 acttgtgtgt aaatttttca ttatctatag gtaaaagcac acttggaatt agcaatagat
4621 gcaatttggg acttaactct ttcagtatgt cttatttcta agcagagtat ttagtttggt
4681 tagtaattac taaacactga gaactaaatt gcaaacacca agaactaaaa tgttcaagtg
4741 ggaaattaca gttaaatacc atggtaatga ataaaaggta caaatcgttt aaactcttat
4801 gtaaaatttg ataagatgtt ttacacaact ttaatacatt gacaaggtct tgtggagaaa
4861 acagttccag atggtaaata tacacaaggg atttagtcaa acaatttttt ggcaagaata
4921 ttatgaattt tgtaatcggt tggcagccaa tgaaatacaa agatgagtct agttaataat
4981 ctacaattat tggttaaaga agtatattag tgctaatttc cctccgtttg tcctagcttt
5041 tctcttctgt caaccccaca cgcctttggc acaatgaagt gggtaacctt tatttccctt
5101 ctttttctct ttagctcggc ttattccagg ggtgtgtttc gtcgagatgc acgtaagaaa
5161 tccatttttc tattgttcaa cttttattct attttcccag taaaataaag ttttagtaaa
5221 ctctgcatct ttaaagaatt attttggcat ttatttctaa aatggcatag tattttgtat
5281 ttgtgaagtc ttacaaggtt atcttattag taaaattcaa acatcctagg taaaaaaaaa
5341 aaaaggtcag aattgtttag tgactgtaat tttcttttgc gcactaagga aagtgcaaag
5401 taacttagag tgactgaaac ttcacagaat agggttgaag attgaattca taactatccc
5461 gaagacctat ccattgcact atgctttatt taaaaaccac aaaacctgtg ctgttgatct
5521 cataaataga acttgtattt atatttattt tcattttagt ctgtcttctt ggttgctgtt
5581 gatagacact aaaagagtat tagatattat ctaagtttga atataaggct ataaatattt
5641 aataattttt aaaatagtat tcttggtaat tgaattattc ttctgtttaa aggcagaaga
5701 aataattgaa catcatcctg agtttttctg taggaatcag agcccaatat tttgaaacaa
5761 atgcataatc taagtcaaat ggaaagaaat ataaaaagta acattattac ttcttgtttt
5821 cttcagtatt taacaatcct tttttttctt cccttgccca gacaagagtg aggttgctca
5881 tcggtttaaa gatttgggag aagaaaattt caaagccttg taagttaaaa tattgatgaa
5941 Ccaaatttaa tgtttctaat agtgttgttt attattctaa agtgcttata tttccttgtc
6001 atcagggttc agattctaaa acagtgctgc ctcgtagagt tttctgcgtt gaggaagata
6061 ttctgtatct gggctatcca ataaggtagt cactggtcac atggctattg agtacttcaa
6121 atatgacaag tgcaactgag aaacaaaaac ttaaattgta tttaattgta gttaatttga
6181 atgtatatag tcacatgtgg ctaatggcta ctgtattgga cagtacagct ctggaacttg
6241 cttggtggaa aggactttaa tataggtttc ctttggtggc ttacccacta aatcttcttt
6301 acatagcaag cattcctgtg cttagttggg aatatttaat tttttttttt ttttaagaca
6361 gggtctcgct ctgtcgccca ggctggagtg cagtggcgca atctcggctc actgcaaact
6421 ccgcctcccg ggttcacgcc attctcctgc ctcagcctcc cgagtagctg ggactacagg
6481 cgcccgccat cacgcccggc taatcttttg tatttttagt agagatgggg tttcaccgtg
6541 tgccaggatg gtctcaatct cctgacatcg tgatctgccc acctcggcct cccaaagtgc
6601 tgggattaca ggagtgagcc accgcgcccg gcctatttaa atgtttttta atctagtaaa
6661 aaatgagaaa attgtttttt taaaagtcta cctaatccta caggctaatt aaagacgtgt
6721 gtggggatca ggtgcggtgg ttcacacctg taatcccagc actttggaag gctgatgcag
6781 gaggattgct tgagcccagg agttcaagac cagcctgggc aagtctcttt aaaaaaaaca
6841 aaacaaacaa acaaaaaaat taggcatggt ggcacatgcc tgtagtccta gctacttagg
```

### Test 12 gRNAs with SaCas9

SaCas9 (3.2 kb) can easily fit into AAV vectors. A total of 12 gRNAs targeting different loci in the intron 1 of the human albumin gene were tested (see below). After transfecting human hepatocytes (HepG2 cell line or Huh-7 cell line) with plasmids encoding SaCas9 and each candidate gRNA, the cleavage activity at the target locus was measured through sequencing.

SaCas9 gRNA (PAM sequence is bolded and yellow highlighted)

1.
(SEQ ID NO: 1)
ttgctgttgatagacactaa aagagt
2.
(SEQ ID NO: 2)
tcacagaatagggttgaaga ttgaat
3.
(SEQ ID NO: 3)
atccca aagacctatccattgcacta
4.
(SEQ ID NO: 4)
attctt ctgtttaaaggcagaagaaat
5.
(SEQ ID NO: 5)
tagtaaactctgcatcttt aaagaat
6.
(SEQ ID NO: 6)
aaggaaagtgcaaagtaact tagagt
7.
(SEQ ID NO: 7)
atccat tgcactatgctttatttaaa
8.
(SEQ ID NO: 8)
aatccat ttttctattgttcaactttt
9.
(SEQ ID NO: 9)
attcat aactatcccaaagacctatcc
10.
(SEQ ID NO: 10)
ctgtgctgttgatctcataaa tagaa
11.
(SEQ ID NO: 11)
atcctg agtttttctgtaggaatcag
12.
(SEQ ID NO: 12)
attctt ctgtttaaaggcagaagaaa

None of these 12 gRNAs mediated detectable cleavage at the albumin intron 1 locus. The disclosure includes the use of gRNAs having at least 90%, 95%, 98% or 99% nucleotide sequence identity to one of SEQ ID Nos. 1-12.

### Test 3 gRNAs with SpCas9

Due to its large size (4.1 kb), it is difficult to fit SpCas9 into AAV vectors although the PAM sequence of SaCas9 is 5′-NNGRRT-3′, while the PAM sequence of SpCas9 is 5′-NGG-3, i.e., there are more PAM sequence options in the target locus for SpCas9. Thus, 3 gRNAs (different than the previous 12 gRNAs) were tested (see below) in human hepatocytes (Huh-7 cell line). Cas9-sgRNA plasmid was transfected into Huh7 cells and 24-48 hours later cells were pooled for genomic DNA extraction. One gRNA (sgRNA 3) showed efficient cleavage activity at the target locus.

SpCas9 gRNA

To clone gRNA into the Bsal-linearized vector, the joint sequence is needed. A “CACC” was added into the 5′ of the sense strand and a “AAAC” was added into the 5′ of the antisense strand.

1.
gRNA1:
(SEQ ID NO: 13)
5′-CACCGACTGAAACTTCACAGAATA-3′
2.
gRNA2:
(SEQ ID NO: 14)
5′CACCGTAGTGCAATGGATAGGTCTT-3′
3.
gRNA3:
(SEQ ID NO: 15)
5′CACCGATTTATGAGATCAACAGCAC-3′

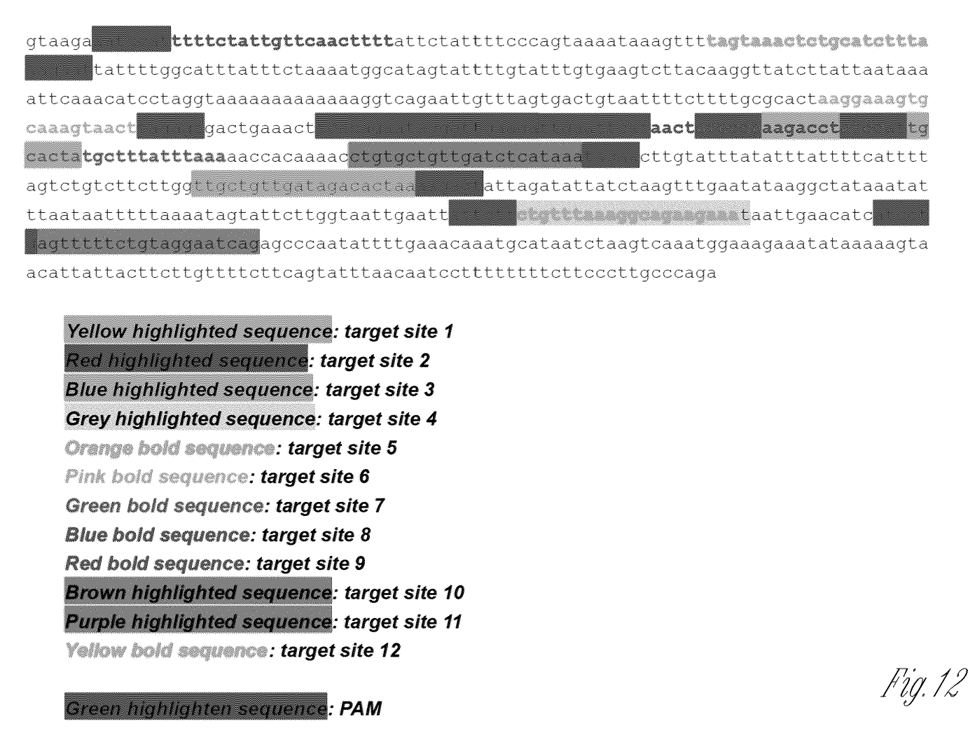

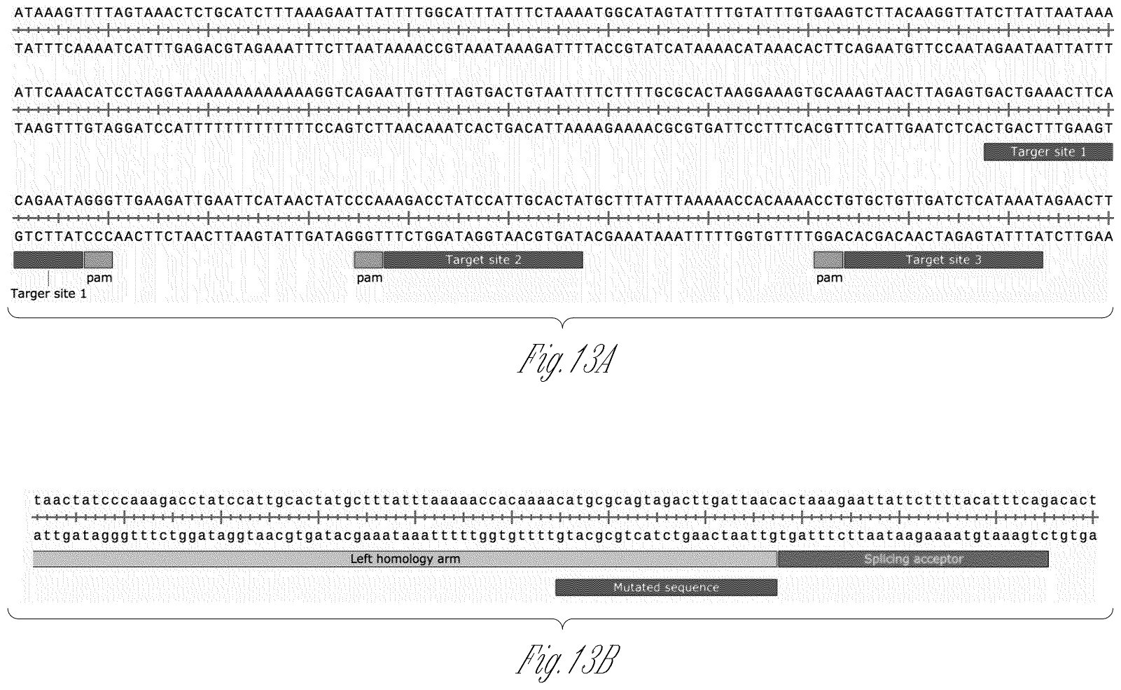

The target site for these 3 gRNAs are shown in FIG. 13A. The PAM sequence adjacent to each target site is also indicated. The disclosure includes the use of gRNAs having at least 90%, 95%, 98% or 99% nucleotide sequence identity to one of SEQ ID Nos. 13-15.

### Off-Target Analysis

Traditional off-target analysis involves 2 steps: 1) predict potential off-target sites through in silico tools; 2) sequence the predicted sites. The in silico tools identify possible off-target sites across the genome and pinpoint the location of mismatches based on the sequences of the genome and gRNA. Although off-target prediction algorithms have been improved over time, their genome-wide search criteria are not exhaustive. Therefore, this method is intrinsically biased. In contrast, GUIDE-seq relies on the integration of a double-stranded oligodeoxynucleotides tag into the double strand break created by Cas9, and then the whole genome is searched for these tags. In this way, off-target sites can be identified.

To assess the off-target effects of SpCas9 and the gRNA identified, an unbiased off-target analysis method, Guide-seq (Tsai 2015), was used. When tested in human hepatocytes (Huh-7 cells), no off-target cleavage was identified.

### In Vitro Pharmacology Studies

After identification of a gRNA that can mediate efficient cleavage at a target locus, it is confirmed that the therapeutic transgene can be inserted and expressed in human hepatocytes. Therefore, a dual vector system was prepared: one vector encoding SpCas9 under the control of a liver-specific promoter, and the other vector encoding gRNA, homology arms and donor cDNA. Normally, the homology arm is the same as the gRNA sequence and the target locus (FIG. 13B).

To avoid continuous cutting, in the left homology arm of the donor construct (where it matches the gRNA sequence and the PAM sequence), mutations were introduced. The original sequence (5′-CCTGTGCTGTTGATCTCATAAAT-3′) (SEQ ID NO:28) was changed into 5′-CATGCGCAGTAGACTTGATTAAC-3′ (SEQ ID NO:29). Then, after homology-directed repair, the homology arm together with these mutations are integrated into the human DNA sequence. Therefore, the target locus is changed, and can not be recognized by Cas9.

The two plasmids are transfected into human hepatocytes, and positive clones are selected. PCR is performed to amplify the target locus and sequencing is conducted to confirm the successful insertion of the therapeutic transgene. Then, enzyme assays are performed with cell lysates and medium to determine the enzyme activity.

### Method to Mutate One of the Homology Arms

As discussed above, to decrease subsequent events catalyzed by Cas9, sequences surrounding the insertion site are analyzed. In one embodiment, only one of the homology arms is mutated, e.g., either the right or the left arm. If the insertion site is within the coding region, silent mutations are introduced, e.g., every 3 bp, so as not to introduce an amino acid substitution. If the insertion site is in an intron (or other non-coding region), mutations may be introduced every 1, 2 or 3 bp. Thus, if a target sequence is about 21 to 25 bp, there are about 7 to 8 mutations

For intron 1 of the human albumin gene, an example of a mutated left arm is as follows:
Left arm (mutated sequence is yellow highlighted):

(SEQ ID NO: 16)
Cgtttgtcctagcttttctcttctgtcaaccccacacgcc
tttggcacaatgaagtgggtaacctttatttcccttcttt
ttctctttagctcggcttattccaggggtgtgtttcgtcg
agatgcacgtaagaaatccatttttctattgttcaacttt
tattctattttcccagtaaaataaagttttagtaaactct
gcatctttaaagaattattttggcatttatttctaaaatg
gcatagtattttgtatttgtgaagtcttacaaggttatct
tattaataaaattcaaacatcctaggtaaaaaaaaaaaaa
ggtcagaattgtttagtgactgtaattttcttttgcgcac
taaggaaagtgcaaagtaacttagagtgactgaaacttca
cagaatagggttgaagattgaattcataactatcccaaag
acctatccattgcactatgctttatttaaaaaccacaaaa
catgcgcagtagacttgattaac
Original sequence:
(SEQ ID NO: 17)
cctgtgctgttgatctcataaat
Mutated sequence:
(SEQ ID NO: 18)
catgcgcagtagacttgattaac
Right arm:
(SEQ ID NO: 19)
agaacttgtatttatatttattttcattttagtctgtctt
cttggttgctgttgatagacactaaaagagtattagatat
tatctaagtttgaatataaggctataaatatttaataatt
tttaaaatagtattcttggtaattgaattattcttctgtt
taaaggcagaagaaataattgaacatcatcctgagttttt
ctgtaggaatcagagcccaatattttgaaacaaatgcata
atctaagtcaaatggaaagaaatataaaaagtaacattat
tacttcttgttttcttcagtatttaacaatcctttttttt
cttcccttgcccagacaagagtgaggttgctcatcggttt
aaagatttgggagaagaaaatttcaaagccttgtaagtta
aaatattgatgaatcaaatttaatgtttctaatagtgttg
tttattattctaaagtgcttatatttccttgtcatcaggg
ttcagattctaaaacagtgc

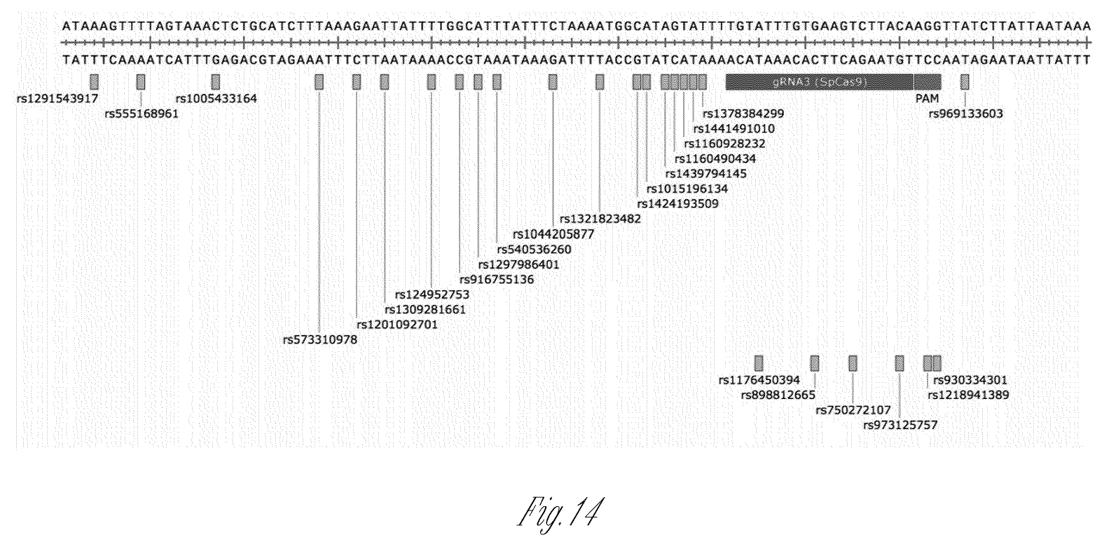

Polymorphisms at the target locus are shown in FIG. 14. The intron 1 of the albumin gene has 709 bp and has 164 polymorphisms. There are 6 polymorphisms at the target locus.

Since intron 1 of human albumin is polymorphic, the sequence of some gRNAs and/or homology arms may be tailored to the genotype of the recipient.

The disclosure includes the use of homology arms having at least 60%, 70%, 75%, 80%, 85%, 90%, 95%, 98% or 99% nucleotide sequence identity to one of SEQ ID Nos. 16 or 19.

### Example 2

There are 70 genetically distinct lysosomal diseases, the majority of which cause severe neurological deficits. Validated surrogate endpoints or biomarkers are critically important to accelerate approvals for gene therapy, by providing a more rapid and easier detection of efficacy than clinical outcomes. However, there are currently no surrogate endpoints or biomarkers that can predict long-term clinical benefit from gene therapy for lysosomal diseases. in GM1-gangliosidosis and GM2-gangliosidosis, chitotriosidase (chito) enzyme activity was one of the few analytes out of approximately 200 screened that appeared to relate to the most severe phenotypes of gangliosidoses. However, no clinical laboratories were positioned to pursue a more rigorous evaluation. Moreover, chito has never been evaluated as a biomarker of gene therapy efficacy.

To investigate CSF chito levels as a surrogate endpoint for clinical trials with gene therapy, this study aimed to (1) validate chito levels for important clinical outcomes in patients with lysosomal diseases and (2) assess the ability of chito to detect effective gene therapy in murine models of lysosomal diseases.

### Methods

A method of quantifying 4-methylumbelliferyl-β-D-N,N′,N″-triacetylchitotrioside in the CSF was developed under conditions comparable with concurrent measurements in serum. A total of 134 CSF and serum specimens were collected from 34 patients with the lysosomal diseases GM1-gangliosidosis (n=8), GM2-gangliosidosis (n=12), Gaucher disease (n=2), mucopolysaccharidoses (MPS, n=11), and multiple sulfatase deficiency (n=1). Gene therapies for three lysosomal diseases were studied in mice with GM1-gangliosidosis, GM2-gangliosidosis, or MPS type I (MPS I). For each disease, CNS tissues were collected from heterozygotes, untreated mice, and treated mice.

### Results

Chito levels in the CSF were significantly higher in patients with gangliosidoses compared to MPS, suggesting distinctive neuroinflammation between the diseases: GM1-gangliodosis vs MPS (p<0.0001); GM2-gangliosidosis vs MPS (p<0.0001). CSF chito levels were higher in patients with the more severe phenotypes compared to milder phenotypes in GM1-gangliosidosis and GM2-gangliosidosis. Furthermore, higher CSF chito levels were significantly associated with higher neurological impairment in patients with GM1-gangliosidosis, GM2-gangliosidosis, and MPS (p=1.12*10−5, R2=0.72). In the CNS tissue for mice with GM1-gangliosidosis and MPS I (affected animals), high chito significantly correlated with low lysosomal enzyme activity (R2=0.86). Moreover, MPS I mice treated with the CNS-effective, PS gene editing system had lower chito levels in the CNS compared to untreated mice (p=0.0004).

### Conclusions

These results provide for the use of CSF chito to measure the therapeutic effect and response to gene therapy in clinical trials for multiple lysosomal diseases. Thus, chito may be a surrogate endpoint. CSF chito may also be a valuable tool for clinical trial enrichment by objectively differentiating between the phenotypes of a lysosomal disease.

### Example 3

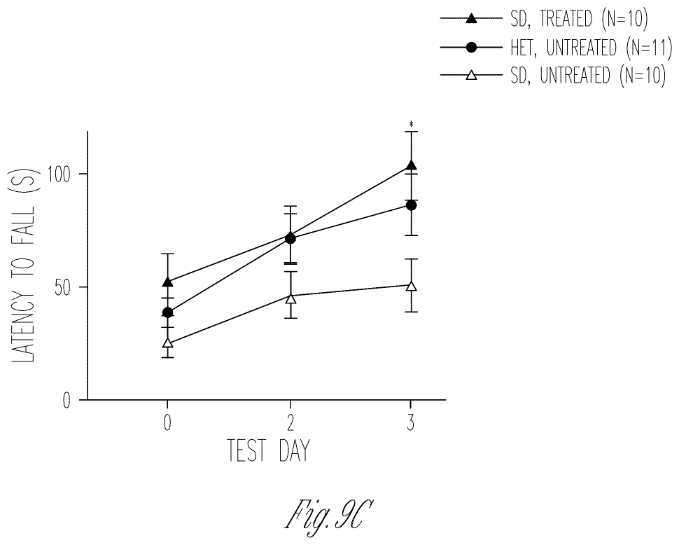

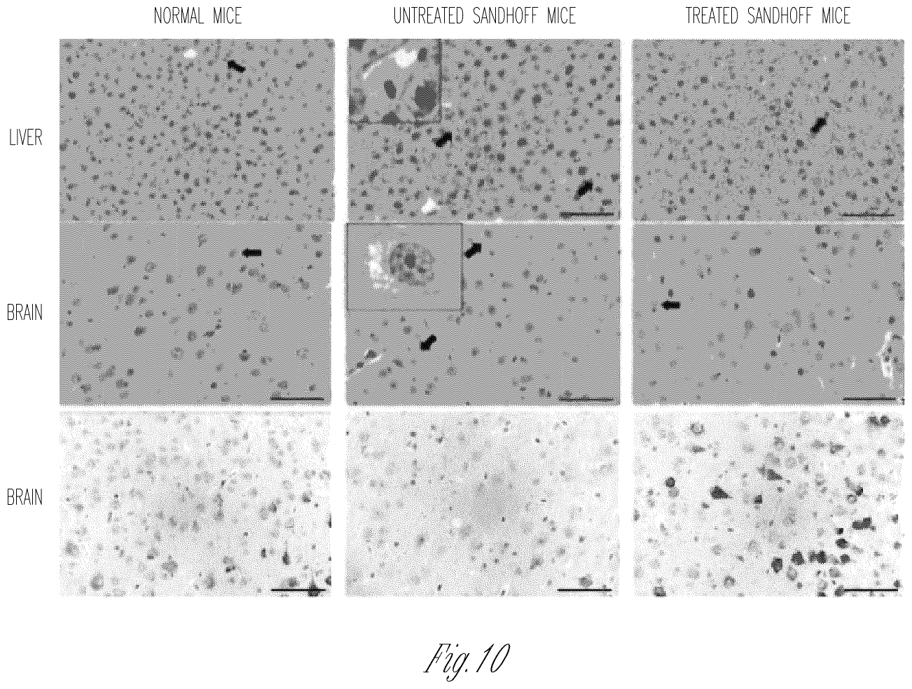

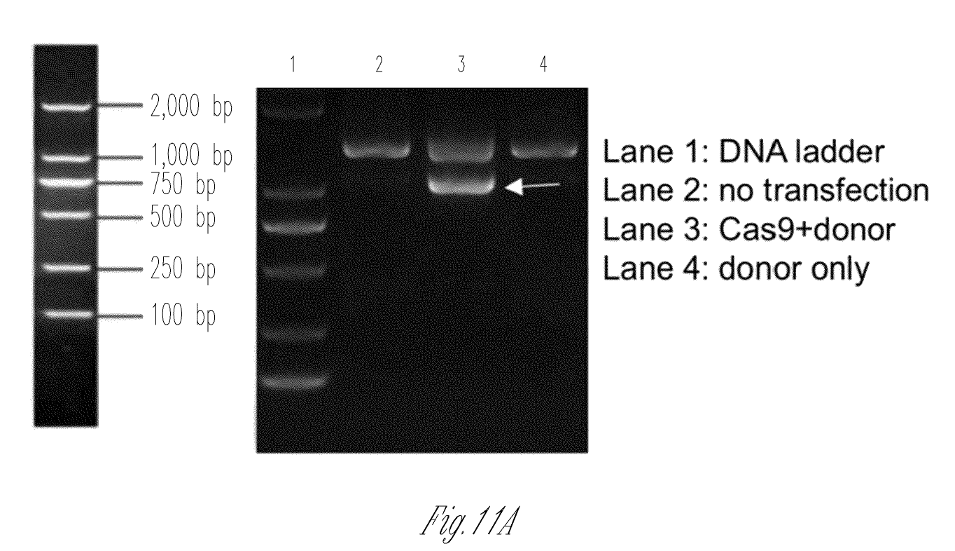

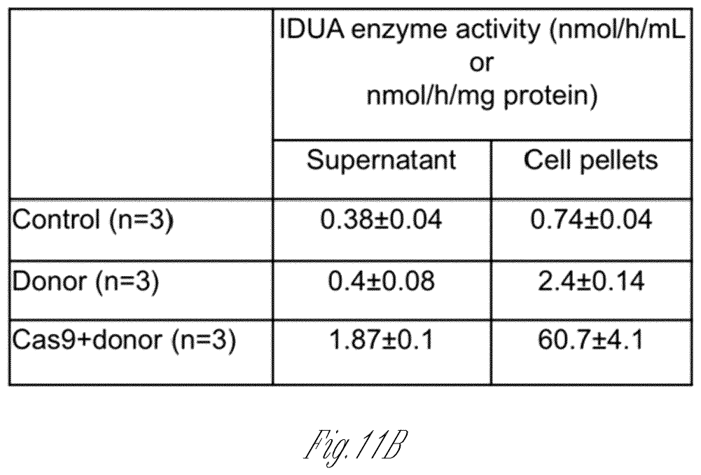

The sgRNA3 (see Example 1) was cloned into the donor plasmid encoding homology arms and IDUA cDNA. The donor plasmid and the plasmid encoding TBG promoter and SpCas9 were cotransfected into HepG2 cells. After extracting genomic DNA from pooled cells, nested PCR were performed with two sets of primers (FIG. 11A). The successful insertion into the target locus was confirmed by sequencing the amplicons. Additionally, the cell pellets and supernatants of cells cotransfected with Cas9 and donor plasmids had significantly higher enzyme activities (FIG. 11B). These results showed that PS Gene Editing efficiently inserted the therapeutic cDNA into the target locus.

Sequencing results at the human albumin locus to confirm integration:

(SEQ ID NO: 30)
Gcagccaatgaaatacaaagatgagtctagttaataatct
acaattattggttaaagaagtatattagtgctaatttccc
tccgtttgtcctagcttttctcttctgtcaaccccacacg
cctttggcacaatgaagtgggtaacctttatttcccttct
ttttctctttagctcggcttattccaggggtgtgtttcgt
cgagatgcacgtaagaaatccatttttctattgttcaact
tttattctattttcccagtaaaataaagttttagtaaact
ctgcatctttaaagaattattttggcatttatttctaaaa
tggcatagtattttgtatttgtgaagtcttacaaggttat
cttattaataaaattcaaacatcctaggtaaaaaaaaaaa
aaggtcagaattgtttagtgactgtaattttcttttgcgc
actaaggaaagtgcaaagtaacttagagtgactgaaactt
cacagaatagggttgaagattgaattcataactatcccaa
agacctatccattgcactatgctttatttaaaaaccacaa
aacctgtgctgttgatctcataaatactaaagaattattc
ttttacatttcagacactagtgctccccctgtcgcccctg
ccgaagccccccacctggtgcaggtggacgccgccagagc
cctgtggcccctgaggcggttctggcggagcaccggcttt
tgcccccctctgccccacagccaggccgaccagtacgtgc
tgtcctgggaccagcagctgaacctggcctacgtgggcgc
cgtgccccaccggggcatcaagcaggtgcggacccactgg
ctgctggaactggtgaccacccggggcagcaccggcaggg
gcctgagctacaacttcacccacctggacggctacctgga
cctgctgcgggagaaccagctgctgcccggcttcgagctg
atgggcagcgccagcggccacttcaccgacttcgaggaca
agcagcaggtgttcgagtggaaggacctggt

Exemplary Cas sequences are as follows:

SpCas9 protein sequence
(SEQ ID NO: 31)
DKKYSIGLDIGTNSVGWAVITDEYKVPSKKFKVLGNTDRH
SIKKNLIGALLFDSGETAEATRLKRTARRRYTRRKNRICY
LQEIFSNEMAKVDDSFFHRLEESFLVEEDKKHERHPIFGN
IVDEVAYHEKYPTIYHLRKKLVDSTDKADLRLIYLALAHM
IKFRGHFLIEGDLNPDNSDVDKLFIQLVQTYNQLFEENPI
NASGVDAKAILSARLSKSRRLENLIAQLPGEKKNGLFGNL
IALSLGLTPNFKSNFDLAEDAKLQLSKDTYDDDLDNLLAQ
IGDQYADLFLAAKNLSDAILLSDILRVNTEITKAPLSASM
IKRYDEHHQDLTLLKALVRQQLPEKYKEIFFDQSKNGYAG
YIDGGASQEEFYKFIKPILEKMDGTEELLVKLNREDLLRK
QRTFDNGSIPHQIHLGELHAILRRQEDFYPFLKDNREKIE
KILTFRIPYYVGPLARGNSRFAWMTRKSEETITPWNFEEV
VDKGASAQSFIERMTNFDKNLPNEKVLPKHSLLYEYFTVY
NELTKVKYVTEGMRKPAFLSGEQKKAIVDLLFKTNRKVTV
KQLKEDYFKKIECFDSVEISGVEDRFNASLGTYHDLLKII
KDKDFLDNEENEDILEDIVLTLTLFEDREMIEERLKTYAH
LFDDKVMKQLKRRRYTGWGRLSRKLINGIRDKQSGKTILD
FLKSDGFANRNFMQLIHDDSLTFKEDIQKAQVSGQGDSLH
EHIANLAGSPAIKKGILQTVKVVDELVKVMGRHKPENIVI
EMARENQTTQKGQKNSRERMKRIEEGIKELGSQILKEHPV
ENTQLQNEKLYLYYLQNGRDMYVDQELDINRLSDYDVDHI
VPQSFLKDDSIDNKVLTRSDKNRGKSDNVPSEEVVKKMKN
YWRQLLNAKLITQRKFDNLTKAERGGLSELDKAGFIKRQL
VETRQITKHVAQILDSRMNTKYDENDKLIREVKVITLKSK
LVSDFRKDFQFYKVREINNYHHAHDAYLNAVVGTALIKKY
PKLESEFVYGDYKVYDVRKMIAKSEQEIGKATAKYFFYSN
IMNFFKTEITLANGEIRKRPLIETNGETGEIVWDKGRDFA
TVRKVLSMPQVNIVKKTEVQTGGFSKESILPKRNSDKLIA
RKKDWDPKKYGGFDSPTVAYSVLVVAKVEKGKSKKLKSVK
ELLGITIMERSSFEKNPIDFLEAKGYKEVKKDLIIKLPKY
SLFELENGRKRMLASAGELQKGNELALPSKYVNFLYLASH
YEKLKGSPEDNEQKQLFVEQHKHYLDEIIEQISEFSKRVI
LADANLDKVLSAYNKHRDKPIREQAENIIHLFTLTNLGAP
AAFKYFDTTIDRKRYTSTKEVLDATLIHQSITGLYETRID
LSQLGGD
SaCas9 protein sequence
(SEQ ID NOs: 32)
KRNYILGLDIGITSVGYGIIDYETRDVIDAGVRLFKEANV
ENNEGRRSKRGARRLKRRRRHRIQRVKKLLFDYNLLTDHS
ELSGINPYEARVKGLSQKLSEEEFSAALLHLAKRRGVHNV
NEVEEDTGNELSTREQISRNSKALEEKYVAELQLERLKKD
GEVRGSINRFKTSDYVKEAKQLLKVQKAYHQLDQSFIDTY
IDLLETRRTYYEGPGEGSPFGWKDIKEWYEMLMGHCTYFP
EELRSVKYAYNADLYNALNDLNNLVITRDENEKLEYYEKF
QIIENVFKQKKKPTLKQIAKEILVNEEDIKGYRVTSTGKP
EFTNLKVYHDIKDITARKEIIENAELLDQIAKILTIYQSS
EDIQEELTNLNSELTQEEIEQISNLKGYTGTHNLSLKAIN
LILDELWHTNDNQIAIFNRLKLVPKKVDLSQQKEIPTTLV
DDFILSPVVKRSFIQSIKVINAIIKKYGLPNDIIIELARE
KNSKDAQKMINEMQKRNRQTNERIEEIIRTTGKENAKYLI
EKIKLHDMQEGKCLYSLEAIPLEDLLNNPFNYEVDHIIPR
SVSFDNSFNNKVLVKQEENSKKGNRTPFQYLSSSDSKISY
ETFKKHILNLAKGKGRISKTKKEYLLEERDINRFSVQKDF
INRNLVDTRYATRGLMNLLRSYFRVNNLDVKVKSINGGFT
SFLRRKWKFKKERNKGYKHHAEDALIIANADFIFKEWKKL
DKAKKVMENQMFEEKQAESMPEIETEQEYKEIFITPHQIK
HIKDFKDYKYSHRVDKKPNRELINDTLYSTRKDDKGNTLI
VNNLNGLYDKDNDKLKKLINKSPEKLLMYHHDPQTYQKLK
LIMEQYGDEKNPLYKYYEETGNYLTKYSKKDNGPVIKKIK
YYGNKLNAHLDITDDYPNSRNKVVKLSLKPYRFDVYLDNG
VYKFVTVKNLDVIKKENYYEVNSKCYEEAKKLKKISNQAE
FIASFYNNDLIKINGELYRVIGVNNDLLNRIEVNMIDITY
REYLENMNDKRPPRIIKTIASKTQSIKKYSTDILGNLYEV
KSKKHPQIIKKG

### REFERENCES

Aronovich et al., PLoS One, 8:e78161 (2013).
Blanz et al., Hum Mol Genet., 17:3437 (2008).
Bradbury et al., Mol. Ther., 21:1306 (2013).
Brunetti-Pierri et al., Mol. Ther., 27:36 (2019).
Cachón-González et al., Mol. Ther., 20:1489 (2012).
Cachón-González et al., Proc. Natl Acad. Sci. USA, 103:10373 (2006).
Calcedo and Wilson, Front Immunol., 4:341 (2013).
Cho et al., Orphanet. J. Rare, Dis., 10:141(2015),
Crawley et al., Mol. Genet. Metab., 89:48 (2006).
Davidoff et al., Blood, 102:480 (2003).
Donsante et al., Science, 317:477 (2007).
Doshi & Arruda, Ther. Adv. Hematol., 9:273 (2018).
Dunder FASEB J., 14:361 (2000).
Eisengart et al., Genet. Med., 20:1423 (2018).
Favaro et al., Hum. Gene Ther., 22:843 (2011).
Finn et al., Blood, 116:5842 (2010).
Goldsworthy & Fransson-Steen, Toxicol. Pathol., 30:97 (2002),
Golebiowski et al., Hum. Gene Ther., 28:510 (2017),
Golebiowski Hum. Gene Ther., 28:510 (2017).
Hacein-Bey-Abina et al., Science, 302:415 (2003).
Harmatz et al., Mol. Genet. Metab., ______:______ (2019).
Hendel et al., Trends Biotechnol., 33:132 (2015).
Hobbs et al., Lancet, 2:709 (1981).
Hockly et al., Brain Res. Bull., 61:469 (2003).
Itakura et al., Biol. Pharm. Bull., 29:1564 (2006),
Jacobs et al., Bone Marrow Transplant., 36:925 (2005).
Jiang et al., Blood, 108:107 (2006).
Karumuthil-Melethil et al., Hum. Gene Ther., 27:509 (2016).
Karumuthil-Melethil et al., Mol Ther., 25:595 (2017),
Kishnani et al., Mol. Genet. Metab., 117:66 (2016).
Kyrkanides et al., Brain Res. Mol. Brain Res., 133:286 (2005).
Laoharawee et al., Mol. Ther., 26:1127 (2018).
Lee et al., FASEB J. 19:1549 (2005).
Lewis et al., WORLD Symposium, San Diego, 2018 February.
Liao et al., Cell, 171:1495 (2017).
MacLachlan et al., Hum. Gene Ther. Clin. Dev., 24:1 (2013).
Maeder et al., Nat. Med., 25:229 (2019).
Maegawa et al., Mol. Genet. Metab., 98:215 (2009).
Martin-Fernandez et al., Nat. Neurosci., 20:1540 (2017).
Matzner et al., Hum. Mol. Genet., 14;1139 (2005).
Matzner et al., Mol. Ther., 17:600 (2009).
Meliani et al., Nat. Common., 9:4098 (2018).
Mingozzi et al., J. Clin. Invest., 111:1347 (2003).
Minghetti et al., J. Biol. Chem., 25:6747 (1986).
Muenzer et al., Mol. Genet. Metab., 126:104 (2019).
Nakai et al., J. Virol., 75:6969 (2001).
Nathwani et al., Blood, 100:1662 (2002).
Nathwani et al., Blood, 107:2653 (2006).
Nathwani et al., Gene Ther., 16:60 (2009).
Nelson et al., Nat. Med., 25:427 (2019).
Nelson et al., Science, 351:403 (2016).
Neufeld & Muenzer, Scriver C R et al (eds), 111:3421(2001).
Nishiyama et al., Neuron., ______:______ (2017).
Ogawa et al., Res. Commun. Chem. Patrol. Pharmacol., 50:435 (1985).
Ohmi et al., Proc. Natl. Acad. Sci. USA, 100:1902 (2003).
Ohmori et al., Sci. Rep., 7:4159 (2017).
Okamoto et al., Sci, Rep., 9:4811 (2019),
Osher et al,, Mol. Genet. Metab., 102:356 (2011).
Osman et al., Hum. Gene Ther., 27:497 (2016),
Ou et al., Mol. Genet. Metab., 111:116 (2014).
Ou et al., Mol. Genet. Metab. 123:105 (2018).
Ou et al., Mol. Genet. Metab. Rep., 8:93 (2016).
Ou et al., Mol. Genet. Metab., 1096:30437 (2018).
Ou et al., Mol. Genet. Metab., 111:116 (2014).
Ou et al., Mol. Ther., 27:178 (2019).
Ou et al., Mol. Ther., In press.
Passini et al., J. Neurosci., 22:6437 (2002).
Polito et al., Hum. Mol. Genet., 19:4871 (2010).
Przybilla et al., Mol. Genet. Metab., Under review.
Ran et al., Nature, 520:186 (2015).
Races et al., Hum. Mol. Genet., 13:1979 (2004).
Rozaklis et al., Exp. Neurol., 230:123 (2011).
Samaranch et al., Hum. Gene Ther. Methods, 27:13 (2016).
Sands and Davidson, Mol. Ther., 13:839 (2006).
Sango et al., Nat. Genet., 11:170 (1995).
Shapiro et al., Mol. Genet. Metab., 122:8 (2017).
Sharma et al., Blood, 126:1777 (2015).
Shin et al., Proc. Natl. Acad. Sci. USA, 72:4435 (1975).
Stone et al., J. Virol., 82:7711 (2008).
Tabebordbar et al., Science, 351:407 (2016).
Tropak et al., Mol. Ther. Methods Clin. Dev., 3:15057 (2016).
Tsai et al., Nat. Biotechnol., 33:187 (2015).
Tsuji et al., Ann. Neurol., 69:691 (2011).
Uniyal et al., Biotech., 9:223 (2019).
Vogler et al., Proc. Natl. Acad. Sci. USA, 102:14777 (2005).
Wang et al., Blood, 133:2745 (2019).
Wang et al., Mol. Ther., 18:118 (2010).
Wang et al., Proc. Natl. Acad. Sci. USA, 2013 Feb. 19; 110(8):2999-3004.
Whitley et al., Birth Defects Orig. Artic. Ser., 22:7 (1986).
Wraith et al., J. Pediatr., 144:581 (2004).
Xue et al., Mol. Genet. Metab., 117:419 (2016).
Yang et al., Nat. Biotechnol., 34:334 (2016).
Zincarelli et al., Mol. Ther., 16:1073 (2008).
Zuo et al., Science, 364:289 (2019).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prevent, inhibit or treat a disease in a human, comprising:
administering to the human an effective amount of i) Cas or an isolated nucleic encoding Cas, and ii) isolated nucleic acid for one or more gRNAs comprising a targeting sequence for a human genomic target and nucleic acid comprising a coding sequence for a prophylactic or therapeutic gene product flanked by homology arms that bind to the human genomic target, or
an effective amount of iii) isolated nucleic encoding Cas and nucleic acid for one or more gRNAs comprising a targeting sequence for a human genomic target, and iv) isolated nucleic acid comprising a coding sequence for a prophylactic or therapeutic gene product flanked by homology arms that bind to the human genomic target,
wherein the expression of the coding sequence in the human prevents, inhibits or treats the disease.

2. The method of claim 1 wherein the disease is mucopolysaccharidosis, a lysosomal storage disease, hemophilia, thalassemia, or sickle cell disease.

3. The method of claim 1, wherein the targeting sequence or homology arms are targeted to an intron.

4. The method of claim 3 wherein the intron is an albumin gene intron.

5. The method of claim 3 wherein the intron is the first intron.

6. The method of claim 1, wherein one or more adeno-associated virus (AAV), adenovirus or lentivirus is/are employed to deliver at least one of i) or ii) or at least one of iii) or iv).

7. The method of claim 6 wherein a first rAAV delivers nucleic acid encoding SpCas9.

8. The method of claim 7 wherein a second rAAV delivers the nucleic acid comprising the targeting sequence and the coding sequence.

9-10. (canceled)

11. The method of claim 1 wherein one or more of the gRNAs target an albumin locus, Rosa26 locus, BCR locus, AAVS1 locus, CCR5 locus, HPRT locus, or alpha fetoprotein locus.

12-13. (canceled)

14. The method of claim 1 wherein the coding sequence encodes iduronidase, beta-globin, iduronate, beta galactosidase, sulfatase, arylsulfatase B, hexM, hexosaminidase A or hexosaminidase B.

15. The method of claim 3 wherein the targeting sequence targets sequences within the first 500, 400, 300, 200, or 100 nucleotides of the intron.

16. The method of claim 1 wherein the Cas comprises Streptococcus pyogenes (SpCas9), Staphylococcus aureus (SaCas9), Streptococcus thermophilus (StCas9), Neisseria meningitidis (NmCas9), Francisella novicida (FnCas9), Campylobacter jejuni (CjCas9), CasX and CasY, Cas12a (Cpf1), Cas14a, eSpCas9, SpCas9-HF1, HypaCas9, Fokl-Fused dCas9, or xCas9.

17. (canceled)

18. The method of claim 1 wherein the nucleic acid comprising a coding sequence for a prophylactic or therapeutic gene product is not operably linked to a promoter.

19. (canceled)

20. The method of claim 1 wherein the gRNA is targeted to a region that is polymorphic.

21. The method of claim 1 wherein at least one homology arm is targeted to a region that is not polymorphic.

22. The method of claim 1 wherein at least one homology arm is targeted to a region that is polymorphic.

23. The method of claim 22 wherein the polymorphism comprises rs1291543917, rs555168961, rs1005433164, rs573310978, rs1201092701, rs1309281661, rs124952753, rs916755134, rs1297986401, rs540536260, rs1044205877, rs1321823482, rs1424193509, rs1015196134, rs1439794145, rs1160490434, rs1160928232, rs1441491010, rs1378384299, rs969133603, rs1176450394, rs898812665, rs750272107, rs973125757, rs1218941389, or rs930334301.

24. The method of claim 1 wherein at least one homology arm is mutated relative to the genomic sequence in the human genomic target.

25. (canceled)

26. A composition comprising a first vector comprising an isolated nucleic encoding Cas9 and a second vector comprising an isolated nucleic comprising sequences for one or more gRNAs comprising a selected human genomic targeting sequence and a selected coding sequence flanked by homology arms that bind to the human genomic target, or a first vector comprising an isolated nucleic encoding Cas9 and an isolated nucleic comprising sequences for one or more gRNAs comprising a selected human targeting sequence and a second vector comprising a selected coding sequence flanked by homology arms that bind to the human genomic target, wherein at least one of the homolog arms is mutated.

27-34. (canceled)

35. A method to decrease Cas9 activity on a nucleic acid template having two homology arms specific for a locus in a mammalian genome, comprising: introducing into a mammalian cell a nucleic acid template having two homology arms each flanking a nucleotide sequence of interest, wherein at least one of the homology arms is mutated, and wherein the cell comprises Cas9 and a gRNA.

36-46. (canceled)
